(19) United States
(12) Patent Application Publication
(10) Pub. No.: US 2005/0175959 A1
(43) Pub. Date: Aug. 11, 2005

(54) System for the controlled delivery of an active material to a dental site

(75) Inventors: Ahron Jodaikin, Kiryat Telstone (IL); Hilary Jodaikin, Kiryat Telstone (IL)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Coll Partners Ltd., Tortola (VG)

(21) Appl. No.: 11/011,007

(22) Filed: Dec. 13, 2004

Related U.S. Application Data
(63) Continuation-in-part of application No. 10/221,465, filed on Jan. 6, 2003.

(30) Foreign Application Priority Data
Mar. 14, 2000 (IL) 135061

(51) Int. Cl. A61C 5/04

(52) U.S. Cl. 433/80; 433/215

(57) ABSTRACT

A system for the strategic controlled delivery of materials to the dental surfaces of the intraoral cavity, in particular materials having a desired or predetermined activity with respect to such dental surfaces for example by forming a specific controlled microenvironment. In particular, the system enables delivery of fluoridizing and other agents to interproximal (approximal) sites among others, to enable inter alia the prevention, treatment, diagnosis, elimination or retardation of dental disease and sensitivity and to provide cosmetic changes. A novelty of this invention is the development of a single or multiphase delivery system in single, bi or multi layer or bi or multi location form or continuation thereof for the optimal delivery of a material or materials to at least one specific dental site with or without other optimal and/or physical intervention.

### CROSS-REFERENCE TO RELATED APPLICATIONS

This application is a continuation-in-part of U.S. application Ser. No. 10/221,465, filed on Jan. 6, 2003, the disclosure of which is incorporated herein by reference.

### FIELD OF THE INVENTION

The present invention relates generally to the strategic delivery of a material or materials to the dental surfaces of the oral cavity, in particular at least one material having a desired or predetermined activity with respect to such dental surfaces. More particularly, the present invention is directed at the delivery of fluoridising and other agents to pits and fissures, interproximal (approximal) zones and to carious sites, among others, to enable inter alia the prevention, treatment, diagnosis, cosmetic treatment, elimination or retardation of diseases, more particularly of dental caries, erosion and sensitivity.

### BACKGROUND OF THE INVENTION

All publications mentioned throughout this application are fully incorporated herein by reference, including all references cited therein.

Teeth are in a constant state of demineralization and remineralization. Thus, incipient caries is a reversible process. The decline in the incidence of dental caries (demineralization) is the result of a tilt toward remineralization domination, primarily due to the use of fluoride (Paine, M. L. et al JADA 129: 69-77, (1998). However, this decline in dental caries has now reached a plateau and dental caries continues to affect a significant segment of the population (Featherstone, J. D. B., CDA J 31:129 2003), especially at plaque stagnation sites. These sites may be occlusal pits and fissures, buccolingual or interproximal. The latter two can involve the enamel or root dentin or cementum surfaces. As occlusal and buccolingual caries occur at accessible tooth surfaces, they are easier to treat and are more readily prevented with oral hygiene, fluoride (see Seppa, L. Compendium Sp Is. 20:18-26, 1999;Seppa, L. and Tolone, T. Scand. J. Dent Res 98:102, 105,1990; Osvik, A. et al Dent Norske Tann. Tidende 104:4-6, 1994 and Seppa L. et al Caries Res 29: 327, 3301995) and pit and fissure sealants. On the other hand, interproximal caries are not only difficult to prevent, but they are also difficult to diagnose (Bader, J. D. and Brown, J. P. JADA 124:48-50,(1993), Ramanathan, G. P. J. Clin. Pediatr. Dent 23:235, 245, 1999) and treat, while the prognosis of interproximal restorations is also poor. (Mjor I. A. Oper Dent 10:88-92,1985). Furthermore, in order to gain access to small interproximal cavities the dentist may have to drill away relatively large quantities of healthy tooth.

This difficult, inaccessible region has been recognized as a problem for more than half a century. Approaches have ranged from grinding of the interproximal surfaces to make them self-cleansing and thus caries “immune” (Mjor, I. A. Quintessence Int.29: 600-602, 1998) to flossing between the teeth which requires fastidious patient compliance and smooth surfaces. The inclusion of fluoride (U.S. Pat. No. 4,638,823) and other agents in dental floss (U.S. Pat. No. 5,875,799) and other electric (U.S. Pat. No. 5,579,786) or mechanical and chemical devices (U.S. Pat. Nos. 4,576,190, 4,638,823 and 5,373,599) does not appear to have significantly reduced interproximal caries. There is thus, a need to develop a technique or device which overcomes these limitations and the disadvantages of flossing between the teeth.

Laboratory studies have shown that specific laser treatment could inhibit subsequent caries-like progression by the laser's modifying the composition of the tooth mineral (see Powell G. L. et al SPIE 1880:188-192, 1994; Kantorowitz , Z et al JADA 129: 585, 1998; Westerman, G. H. et al J Clin Laser Med Surg 20:257-262 2002; JDB Featherstone, J. D. B. JADA 131:887-899 2000; Kawasaki, K. et al. Arch Oral Biol 45:797-804 2000; Fried, D. et al. J Biomed Optics 6:231-238 2001). Lasers have also been used in the presence of topical fluoride agents ( Westerman, G. H. et al J Clin Laser Med Surg 20:257-262 2002) and to attach or sinter synthetic hydroxyapatite to enamel (Stewart, L. et al. Oper Dent 10:2-5 1985). These techniques have not always been found to be advantageous (Apel, C. et al, Lasers in Surg & Med 30:337-341 2002; Apel, C et al. Caries Res 37:34-37 2003) nor are they widely used clinically. The strategic placement of agents as described in this invention in conjunction with laser or other applications could be used to modify tooth material or to deposit advantageous agents at interproximal surfaces, pits and fissures, carious sites and root surfaces among others. Examples are deposits such as long term pH controlled fluoride reservoir in various forms of CaF₂ (see Ogaard, B. Caries Res 35: S1:40-44; 2001, Chow, L. C. et al Caries Res 36:136-141, 2002) or favorable modifications by the inclusion of elements (such as fluorine, molybdenum, vanadium and strontium, see Ostrom, C. A. in The Biological of Dental Caries, Menaker, L. ed. p. 249, Harper and Row, 1980), chemicals (such as amorphous calcium carbonate, amorphous calcium phosphate, iron oxide, zinc oxide, silicic acid, and thymol) and crystals (such as aragonite, brushite, calcite, dahltite, ferrihydrite, fluoroapatite, hydroxyapatite, lepidocrocite, magnetite, octocalcium phosphate, vaterite and whitlockite). In order to accomplish this, these types of agents or elements will need to be at the appropriate surface in an appropriate form for applicable laser application and/or other light or heat applications.

Although dental floss can be used to apply varnish interproximally, the effectiveness of this application and other paint on applications has not been fully endorsed (Beltran-Aguilar, E. D. et al JADA 131:589-596, 2000); nor is this a targeted micro dose method which provides precise measurable amounts. Furthermore, another imprecise application, the APF gel tray application, which has FDA and ADA recognition, has not been found to be efficient in applying fluoride into interproximal regions. (see Guo, M. K. et al. J Dent Res 68:496-498, (1986); Seppa, L. Caries Res 29:327-330,(1995), Seppa, L. Compendium Sp. Is. 20:18-26 (1999), Schuller, A. A. and Kalsbeek, H. Caries Res 37:172 (2003)). The high incidence of interproximal caries is due to the fact that current preventive regimes barely manage to reach the surfaces between adjacent teeth in order to take full advantage of fluoride and other ions which can effectively mineralize or remineralize teeth or improve their ability to resist dental caries. (see Poole, D. F. G. and Silverstone, L. M. in Hard Tissue Growth, Repair and Remineralisation pp 35-52 Ciba Foundation Symposium No. 11, Elsevier Scientific Publishing Company, 1973; Legler D. W. and Menaker, L. pp. 211-225 and (Ostrom C. A. pp. 445-460 in The Biological Basis of Dental Caries, Menaker L., Harper & Row, 1980); Winston, A. E. and Bhaskar S. N. JADA 129:1579-1587, 1998, Achievements in (US) Public Health, 48(41):933, 1999; Kashket S, J Clin Dent 10 Sp Is:56-64, 1999; Zero, D. J. Clin Dent 10 Sp Is: 74-85 1999; Chow, L. C. and Vogel, G. L. Oper Dent 6:27-38 2001; Donly, K. J. et al. JADA 130:817 1999; Featherstone, JDB. JADA 131:887-899-, 2000).

Fluoride mouth rinses have also been introduced to reduce dental caries by remineralizing teeth and more recently attempts have been made to reduce the concentration of fluoride and improve the remineralization properties.(Wefel, J. S. and Harless, J. D. J. Dent Res. 66(11):1640-1643, 1987; Chow, L. C. et al Caries Res. 36 : 136-141, 2002). One approach has been the introduction of a two or multi solution rinse by possibly raising the ambient fluoride produced by the greater loosely bound fluoride deposition in the form of CaF₂ or similar deposits (see U.S. Pat. Nos. 4,083,955, 4,397,837, 4,837,007, Nos. 5,145,668 and 5,476,647) and reduce or eliminate the interference of saliva, or produce an advantageous initial delay (U.S. Pat. No. 5,891,448). One example is the introduction of a cationic agent prior to or simultaneously to the introduction of the active agent (U.S. Pat. No. 4,837,007). Another example is a reactive multicomponent composition of an admixture of a stable, non-toxic soluble calcium salt and a soluble calcium complexing (chelating) agent with a stable, non-toxic soluble fluoride compound, a buffer and one or more non-interfering carriers. The components of this admixture are mixed in an aqueous environment resulting in a controlled precipitation of calcium fluoride and then promptly applied to the tooth surface in the form of mouth rinses, dentifrices, gels and chewable tablets. It should be noted that the first and second components may be in contact as in a chewable tablet provided the product is dry until use as this will prohibit mixing of the components in an aqueous environment (Chow, L. C. et al. J Dent Res 79:991-995, 2000). U.S. Pat. No. 6,159,449 emphasizes that the formation of calcium fluoride removes free fluoride anions from the aqueous composition used to remineralize and/or mineralize teeth thereby avoiding the premature formation of calcium fluoride. This above mentioned patent (U.S. Pat. No. 6,159,449) is based on the finding that in an aqueous composition the presence of a particular amount of at least one water soluble or partially water soluble magnesium compound can significantly delay the reaction between a water soluble or partially water soluble calcium compound and a water soluble fluoride compound to form calcium fluoride if the magnesium and calcium compounds are combined prior to being combined with fluoride. Thus, under appropriate conditions, the calcium and magnesium compounds should be first combined and then fluoride ions can be introduced without initial loss of free fluoride anions. Improved anticaries effects have been reported where calcium phosphate and fluoride are combined from a dual chambered toothpaste (see Zhang Y.P. et al J. Clin. Dent 14: 23-28, 2003). Although strips have been used to achieve sustained intra-oral contact by utilizing a second layer to provide adhesive properties (see WO 9962472 and U.S. Pat. No. 6,514,484) these patents as well as other studies and patents listed above and other patents, (U.S. Pat. Nos. 5,460,803, 5,476,647 and 6,159,449) appear to optimize fluoride application and remineralization., they have not provided for a single or multi-phase delivery system in single, multi-layer or multi-location form for the strategic delivery of an agent or agents to a specific dental surface such as interproximal sites.

An attempt to overcome some of these mechanical limitations is U.S. Pat. No. 3,754,322 which describes a device which comprises two distinct parts: a thin strip carrier and a thin caries treatment section which continually dissolves when wet with saliva. However the area of contact between the treatment section and the interproximal site would continually diminish and this device would not enable chemical control of the rate of release of the caries treatment material but would merely dissolve when wet with saliva. The system also comprises a carrier strip and a detachable uniform treatment, the strip being used to position the treatment section in place between teeth after which the carrier is removed leaving only the treatment section in the oral cavity which has limited interproximal tooth surface contact and little retentional properties for physical fixation onto the entire interproximal site for at least a predetermined time period correlated to the delivery of a predetermined portion of a material or materials to said site. Furthermore, said device fails to create a specific controlled micro-environment which isolates the space to be filled and controls conditions, for example, by excluding elements (such as magnesium, which has been associated with increased caries susceptibility (Ostrom C. A. in the Biological Basis of Dental Caries, Menaker L. 445-460, Harper & Row, (1980)) and molecules (such as salivary proteins that inhibit mineral nucleation or that may interfere with optimal remineralization or mineralization (Moreno E C et al Calcif Tissue Int 28:7-16 (1979); Boackle, R. J. and Suddick, R. P. in The Biological Basis of Dental Caries, Menaker L. p. 119, Harper & Row, (1980)). Another controlled micro-environment function can be to create and maintain a high local supersaturation of appropriate ions, such as calcium and phosphate, at optimal levels and with optimal pH in order to encourage mineral nucleation as well as other small molecules such as arginine (see Kleinberg, I. Crit. Rev in Oral Biology and Med. 13, 108-125, 2002). The disadvantage of current topical fluoride applications are toxicity, dilution, buffering effects and interference by saliva, the lack of ability to reach into all susceptible sites, especially interproximally (Guo M. K. et al. J. Dent. Res. 68:496-498, 1989), failure to penetrate through the depth of plaque and the need for relatively frequent applications or extended time spans. These failures are primarily governed by the lack of time that the topical fluoride can be held in the mouth and by the potential toxicity of swallowing the active agent which is used in a gross form and in relatively imprecise large amounts, even by more advanced methods (U.S. Pat. No. 5,770,182) which are also cumbersome, uncomfortable, time consuming unhygienic and fail to reach interproximal areas. An attempt to overcome some of these problems was reported by Rose K. et al. (J. Dent. Res. IADR Abs. 77:972, 1998). It is felt that the wedges described therein would not physically reach the contact points or all areas of teeth interproximally and they would not be retained for extensive periods in a clinical situation because of physical and chemical considerations. Another approach is U.S. Pat. No. 6,136,297 which also does not deal with directly negotiating the interproximal areas, nor do extensive literature reviews (Rawls, H R Adv. Dent. Res. 5,50-55 1991; Featherstone J. D. B. Community Dent Oral Epidemiol 27:31-40 1999; Kashket, S., J.Clin Dent 10:56-64, 1999;Featherstone J. D. B. JADA 131:887-899 2000; Seppa, L. Compendium Sp Is 20:18-26, 1999;Chow, L. C. and Vogel, G. L. Operative Dent. Supp. 6:27,-38, 2001) refer to this approach. Furthermore, neither orthodontic bands, which hold archwires onto orthodontic brackets and release fluoride (available in the U.S.A. from Ortho-Byte), nor fluoride slow releasing glass devices (Toumba, K. J., Caries Res 35 Supp.1:10-13, 2001) specifically target surfaces such as interproximal sites. Rather, these bands or devices release the fluoride into the saliva.

Referring to FIG. 1, there are primarily three tooth zones that are more susceptible to caries:- the occlusal pits and fissures, marked (A), the contact areas between teeth (B1) of interproximal (approximal) regions marked (B), and the cervical margins, marked (C), which can also include root surfaces. Similarly, interproximal regions (B) can also have caries at root surfaces. Contact points exist between adjacent teeth, but with aging, these points wear to form small areas of contact. Pit and fissure sealants, for example, for sites marked (A) are reasonably effective at these occlusal pits and fissures, but they are technique sensitive, require a dry environment, are relatively costly and time consuming and not always durable. Furthermore, they are not applicable to interproximal regions (B) which are difficult to access for prevention or treatment. Topical fluoride applications (e.g., as in U.S. Pat. Nos. 5,770,182 and 6,136,297) are effective at cervical and root regions which are more prevalent today as geriatric patients have saved many of their teeth with exposed weaker dentin due to gingival recession (Mandel, I D Quintessence Int. 16,81-87 1985). However, there is a need for a more localized precise and targeted means of preventing and treating microscopic cervical and root caries especially when the lesions are more advanced.(see Clarkson, B. H. et al Caries Res 32:357-364, 1998; Mukai, Y. and tenCate, J. M. Caries Res. 36:275-280, 2002, Mukai, Y. et al Caries Res. 35:317-324, 2001; Paine, M. L. et al JADA 129: 69-77, 1998).

Dentinal caries marked (A, B and C) comprises four zones, namely the infected necrotic zone, the infected superficial demineralized zone, the affected deep demineralized zone and the hypermineralized zone (Massler, M. Dental Clinics of North America pp 663-673, 1967). Although bacteria are abundant in the superficial demineralized zone, only on rare occasions are a few bacteria found in the affected deep demineralized zone, which comprises well formed residual tubular matrices. Indeed, the major difference between this zone and sound dentin appears to be depletion of the mineral components. The clinical appearance at this zone is that of dry leathery dentinal structure (Hoffman, S. in The Biological Basis of Dental Caries. Menaker, L. pp 226-246, Harper & Row, 1980) which is the dentin collagen which may be fully or partially denatured. Current dental treatment involves mechanically removing this layer and the more superficial layers using drills and mechanical excavation. Attempts to avoid this invasive and painful technique as well as to prevent pulp exposure include air abrasion, lasers, atraumatic restorative therapy, and chemomechanical caries removal. None of these techniques have been fully accepted clinically because of a series of disadvantages and failures. This further emphasizes the need for effective preventative techniques or alternative treatment regimes, especially a chemically based regime.

U.S. Pat. No. 6,326,022 discloses a disposable elastomeric device for insertion and retention between teeth. An active ingredient such as an odorant or medication contained therein is slowly released from the device into the mouth. The device is not biodegradable, as the device must be removed and disposed of after completion of use. Stem portions that are inserted between the teeth are for retention only, and there is no mention of delivery to approximal regions. Moreover, the stem portions are inserted in the small gap which is under the approximal contact area, not between the contact area itself. In an alternative embodiment, a sleeve is provided for sliding over a tooth, and a larger rectangular panel is wrapped around the tooth such that the wrap extends beyond the contact area and into the gums.

It is known that carious lesions in enamel, dentin and cementum are not uniform. They differ geometrically at the physical level which includes the gross structure of the lesion and the crystal orientations on surfaces of the residual minerals. The chemical profile or distribution of types and concentrations of elements, minerals and organic material can also vary which can affect the transport mechanism for remineralization or mineralization (see Kawasaki, K. et al Caries Res 34:395-403, 2000). The present invention includes the possibility of varying active ingredients and matrices or combinations thereof, “tailor-made” for the above variations.

Initially, an anodyne compound consisting of thymol and zinc oxide fused by heating had been used for building a foundation and lining for a cavity (Kay L W in Drugs in dentistry, 2^{nd} ed. Bristol, J Wright and Sons Ltd., 1972 p. 199). Later, chemomechanical caries removal techniques were introduced (Beeley J A et al Brit Dent J 188:427-430, 2000, U.S. Pat. Nos. 5,975,896 and 5,997,301). However, there remains a need for a chemical device which can be swiftly positioned in a carious lesion (prior, during or after chemical or mechanical treatment thereof) which slowly releases agents to utilize some or all of the dry leathery dental carious structure (instead of requiring the removal of the said matter which increases the chances of pulp exposure) by fixing and sterilizing the carious matter, cause remineralization or mineralization, desensitize the pulp and thereby provide a foundation for restoration or further mechanical or chemical treatment prior to restoration.

This invention is not limited to dental caries, but includes other functions such as desensitization at a tooth furcation where access is often difficult because of the anatomy and cervical fluid flow, as well as whitening/bleaching at tooth contact points or areas, which are places that, although are most often stained, are not directly reachable by current techniques.

An aim of the present invention is to provide a system for the controlled or sustained delivery of one or more materials having a desired or predetermined activity to a desired dental site in the oral cavity, which overcomes the disadvantages of prior art systems.

It is another aim of the present invention to provide such a system that is particularly directed to the contact points/areas of interproximal (approximal) sites, as well as the coronal and cervical (enamel, root dentin and/or cementum) surfaces of said interproximal sites.

It is another aim of the present invention to provide such a system that is particularly directed to the pits and fissure of said dental sites.

It is another aim of the present invention to provide such a system that is particularly directed to the buccolingual surfaces of enamel, dentin and cementum sites.

It is another aim of the present invention to provide such a system that is particularly directed to the carious sites of said dental sites.

It is another aim of the present invention to provide such a system that is particularly directed to sensitive sites of said dental sites.

It is another aim of the present invention to provide such a system that employs at least one matrix as a carrier for the active material.

It is another aim of the present invention to provide such a system that employs at least one matrix to deliver a single phase controlled release pattern or a bi- or multiphase controlled release pattern to deliver at least one agent at an appropriate or optimal time, stage, manner or form.

It is another aim of the present invention to provide such a system that employs at least one bi- or multi-layer or bi- or multi-located matrix to provide a single phase, biphase or multiphase controlled release system.

It is another aim of the present invention to provide such a system that employs at least one matrix which keeps the active material or materials inactive by chemical means such as inhibition or physical separation in order to allow at least one agent to be delivered at an appropriate or optimal time, stage, manner or form.

It is another aim of the present invention to provide such a system in which the matrix or matrices for the active material or materials may be biodegradable, self-degradable, resorbable or non-resorbable or any combination thereof.

It is another aim of the present invention to provide such a system which is particularly adapted for physical fixation onto the dental site, in particular on and/or around the interproximal contact points/areas, for at least a predetermined time period, typically sufficient to enable the controlled or sustained delivery of a required quantity of the active material or materials from the matrix or matrices to the site.

It is another aim of the present invention that the system is shaped to fit over a portion of, or an entire single dental arch, or an upper and lower dental arch.

It is another aim of the present invention to provide such a system in which physical fixation (retention) of the at least one matrix is by way of a physical property of the matrix or matrices, in particular wherein the matrix or matrices comprises a hydrophilic polymer which softens and swells in situ by the hydration thereof in the oral cavity after accommodation at the dental site.

It is another aim of the present invention to provide such a system which includes at least one adhering agent.

It is another aim of the present invention to provide such a system in which a specific, controlled micro-environment which selectively excludes at least one element or molecule present in the mouth by way of a physical or chemical property of the matrix or matrices.

It is another aim of the present invention to provide such a system in which a specific, controlled micro-environment which optimizes the delivery of at least one element, molecule or agent to the said dental site. The said element, molecule or agent can be exogenous, from the system, or endogenous, e.g. from the saliva.

It is another aim of the present invention to provide such a system in which physical fixation of the matrix or matrices is primarily by means of a carrier member, which is itself adapted, on the one hand, to accommodate the matrix or matrices and align the same with the dental site, and on the other hand, is adapted for affixing the carrier at the site by virtue of its shape, configuration and elasticity/resilience of the material from which it is made. In particular, such adaptation includes sufficient elasticity and toughness of the matrix or matrices material, which are important criteria when positioning the matrix or matrices between teeth.

It is another aim of the present invention to provide such a system in which physical fixation or placement of the matrix or matrices is primarily by means of a carrier member which is itself adapted on the one hand to accommodate the matrix or matrices in or on the dental site, and on the other hand is also adapted for affixing the carrier at or in the site by virtue of its shape, configuration and elasticity/resilience of the material from which it is made. In particular, at or in a pit and fissure, bucco-lingual interproximal surface or carious sites.

It is another aim of the present invention to provide such a system wherein the matrix or matrices is sufficiently flexible for insertion into the interproximal site, and at the same time is of sufficient toughness to maintain mechanical integrity thereat.

It is another aim of the present invention to provide such a system wherein the matrix or matrices is shaped to facilitate placement at an interproximal site.

It is another aim of the present invention to provide such a system wherein the matrix or matrices is soft enough not to be a source of discomfort within the oral cavity.

It is another aim of the present invention to provide such a system wherein the matrix or matrices can be inserted into the interproximal site to provide interproximal cleaning and/or agents to facilitate mineralization or remineralization.

It is another aim of the present invention to provide such a system wherein the matrix or matrices can be positioned on or adjacent to a tooth surface to provide desensitization of a tooth at said site.

It is another aim of the present invention to provide a system wherein the matrix or matrices can be inserted or positioned at an appropriate site including an interproximal site to provide tooth whitening or bleaching independently or in conjunction with another agent such as fluoride.

It is another aim of the present invention to provide at least one or a combination of chemical agents at a site such as a cervical root surface, interproximal site, occlusal site or carious lesion to facilitate changes by chemical or physical means, such as those induced by a laser to prevent, seal, eliminate, retard, treat or heal dental caries cause desensitization or whitening/bleaching at a tooth surface or surface and/or interface with a restoration or prosthesis.

It is another aim of the present invention to provide any one or combination of a plurality of chemical and other agents that have a desired activity at the dental site, in particular such as to enable inter alia the prevention, treatment, diagnosis, elimination or retardation of dental caries or another dental disease at tooth surfaces or at tooth interfaces with restorations or prostheses.

Other purposes and advantages of the invention will appear as the description proceeds.

### SUMMARY OF INVENTION

The present invention relates to a system for the controlled delivery of at least one material having a predetermined intraoral activity to an interproximal site of at least one dental surface in an oral cavity, comprising at least one matrix containing said at least one material, said at least one matrix being adapted for affixing at said interproximal site for at least a predetermined time period correlated to the delivery of a predetermined portion of said at least one material in one or more phases to said site with or without chemical and/or physical intervention. The interproximal site typically comprises an area of contact and its surrounding surfaces between said dental surface and an adjacent dental surface.

The present invention relates to a system for the controlled delivery of at least one material having a predetermined intraoral activity to an occlusal, or cervical root or carious site of at least one dental surface in an oral cavity, comprising a at least one matrix containing said at least one material, said at least one matrix being adapted for affixing at said site for at least a predetermined time period correlated to the delivery of a predetermined portion of said at least one material in one or more phases to said site with or without other chemical and/or physical intervention.

Preferably, the matrix or matrices are a polymeric material.

Optionally, the matrix or matrices may be substantially self degradable, biodegradable, or resorbable or non- resorbable or any combination thereof.

In one embodiment of the present invention, the matrix or matrices comprise a hydrophilic polymer such as to enable the matrix or matrices to be fixed by swelling in situ by the hydration thereof in the oral cavity after accommodation at said site in particular an interproximal site.

In one embodiment of the present invention, the matrix or matrices comprise a hydrophilic polymer such as to enable the matrix or matrices to be fixed therein by swelling in situ by the hydration thereof in a carious cavity and also seal or adhere to all the cavity surfaces.

In one embodiment of the present invention, the matrix or matrices comprise an adhesive surface such as to enable the matrix or matrices to adhere to a pit and fissure, smooth surface, a carious site, an eroded or abraded surface or another dental site.

In another embodiment of the present invention the system provides a controlled micro-environment which can selectively exclude at least one element or molecule from the saliva and/or optimize the delivery of at least one element molecule or agent to the said dental site from the system or from the saliva.

In another embodiment of the present invention at least one matrix surface can be of an adhesive nature such as to enable the fixing or sticking of the system in situ.

In another embodiment of the present invention the matrix or matrices can be uniform or be composed of more than one matrix type, for example in a bi-layer, bi-location, multi-layer or multi-location form.

In another embodiment of the present invention the matrix or matrices in a single bi-or multiphase system is designed to deliver at least one agent at an appropriate or optional time, stage, manner or form.

In another embodiment of the present invention the entire system or a part thereof can be in a liquid, semi-liquid, colloidal or any form that can be painted onto a said dental surface where it solidifies with no intervention or with chemical or physical intervention such as light application.

In one embodiment of the present invention the polymeric matrix or matrices have a three dimensional form having at least one external surface, wherein at least a portion of said external surface is adapted for contact with at least said interproximal site of said dental surface such as to deliver said material or materials to said site. The matrix or matrices may be in the form of a disc having at least one external substantially flat surface for contact with at least said interproximal site of said dental surface such as to deliver said material or materials to said site. Alternatively, the matrix or matrices may be in the form of a disc having at least one external substantially concave surface for contact with at least said interproximal site of said dental surface such as to deliver said at least one material to said site. Alternatively the matrix or matrices may be in the form of a pellet having at least one external substantially oval surface for contact with at least said interproximal site of said dental surface such as to deliver said at least one material to said site. Alternatively, the matrix or matrices may be, for example, square, round or oval, and attached to a cord at one or both sides, in order to facilitate interproximal placement in a similar manner that dental floss is inserted interproximally. The attached cord may be similar, physically and chemically, to the matrix or matrices, or made from any suitable material. Alternatively, the matrix or matrices may be in the form of a toroidal ring having at least one external substantially annular surface for contact with at least said interproximal site of said dental surface such as to deliver said material or materials to said site.

In another embodiment, the matrix or matrices are in the form of a wedge having at least one external longitudinal surface for contact with at least said interproximal site of said dental surface such as to deliver said material or materials to said site. Alternatively, the matrix or matrices may be in the form of a wedge having at least one winged member at least at one longitudinal end thereof, said wedge having at least one external longitudinal surface for contact with at least said interproximal site of said dental surface such as to deliver said material or materials to said site, and said winged member having suitable contact surfaces for delivering a portion of said material or materials to a portion of said dental surface and an adjacent dental surface mesial and distal to said interproximal site. Alternatively, the matrix or matrices may be in the form of a sphere, dumb-bell or similarly appropriate shape having at least one surface for contact with at least said interproximal site of said dental surface such as to deliver said material or materials to said site. The matrix or matrices of the spherical or dumb-bell shape may be preferably soft for easy interproximal insertion and preferably provides a cleaning effect, releases at least one antimicrobial or cleansing agent and/or at least one remineralizing or mineralizing agent. These systems can be placed interproximally as independent units or linked for example, by a strand or ribbon that connects each unit to one another, in order to facilitate easier and faster positioning along adjacent interproximal sites within a dental arch.

In additional embodiments, at least one inner portion or layer may be in a liquid, gel foam, semi-liquid, colloidal or any other viscous form.

In another embodiment, after biodegradation, resorption or dissolution of the outer portion or portion thereof, the inner portion is released and functions as an oral rinse.

In yet another embodiment, the at least one matrix has a three dimensional form having a first external surface and a second external surface, wherein at least a portion of the first external surface is adapted for contact with at least the interproximal site of the dental surface and wherein at least a portion of the second external surface is adapted for contact with at least the interproximal site of the adjacent dental surface such as to deliver the at least one material.

In yet another embodiment, the at least one matrix is in the form of a disc having opposed first and second external substantially flat surfaces for contact with at least the interproximal site of the dental surface and the adjacent dental surface, respectively, such as to deliver the at least one material to the site.

In yet another embodiment, the matrix is attached to a cord, thereby facilitating interproximal placement.

In yet another embodiment, the at least one matrix is in the form of a disc having opposed first and second external substantially concave surfaces for contact with at least the interproximal site of the dental surface and the adjacent dental surface, respectively, such as to deliver the at least one material to the site.

In yet another embodiment, the matrix is attached to a cord, thereby facilitating interproximal placement.

In yet another embodiment, the at least one matrix is in the form of a pellet having opposed first and second external substantially oval surfaces for contact with at least the interproximal site of the dental surface and the adjacent dental surface, respectively, such as to deliver the at least one material to the site.

In yet another embodiment, the at least one matrix is in the form of a toroidal ring having opposed first and second external substantially annular surfaces for contact with at least the interproximal site of the dental surface and the adjacent dental surface, respectively, such as to deliver the at least one material to the site.

In yet another embodiment, the at least one matrix is in the form of a wedge, having first and second external substantially longitudinal surfaces for contact with at least the interproximal site of the dental surface and the adjacent dental surface, respectively, such as to deliver the at least one material to the site.

In yet another embodiment, the at least one matrix is in the form of a wedge, having at least one pair of winged members at least at one longitudinal end thereof, the wedge having first and second external substantially longitudinal surfaces for contact with at least the interproximal site of the dental surface and the adjacent dental surface, respectively, such as to deliver the at least one material to the site, and the winged members having suitable contact surfaces for delivering a portion of the at least one material to a portion of the dental surface and to a portion of the adjacent surface mesial and distal to the interproximal site.

In yet another embodiment, the at least one matrix is in the form of a sphere, dumb-bell or similarly appropriate shape having at least one surface for contact with at least the interproximal site of the dental surface such as to deliver the at least one material to the site.

In yet another embodiment, the at least one matrix is preferably soft for easy interproximal insertion, preferably provides a cleaning effect which would serve as an alternative or supplement to flossing and releases at least one antimicrobial or cleansing agent and/or at least one remineralizing or mineralizing agent.

In yet another embodiment, at least one unit is linked to another by a strand or band that can be identical in composition to the units' or it can differ in composition.

In yet another embodiment, at least one inner portion or layer may be in a liquid, gel foam, semi-liquid, colloidal or any other viscous form.

In yet another embodiment, after biodegradation, resorption or dissolution of the outer portion or portion thereof, the inner portion is released and functions as an oral rinse.

In yet another embodiment, the system further comprises a suitable support member for fixing the at least one matrix to the site, the support member comprising a peripheral frame portion surrounding a net portion, the frame portion being made from a resilient material capable of enabling the support member to be accommodated at the interproximal site such as to align the net portion therewith, and wherein the net portion is adapted for accommodating the at least one matrix and for enabling the at least one material to be delivered therefrom to the site.

In yet another embodiment, the frame member is in the form of a ring, wherein the member is attached to the inner concave surface of the ring.

In yet another embodiment, the frame member further comprises at least one niche for facilitating gripping of the the frame member to enabling affixing thereof at the interproximal site.

In yet another embodiment, the frame member further comprises at least one loop for facilitating gripping of the the frame member to enabling affixing thereof at the interproximal site.

In yet another embodiment, the support member is made from any suitable material including natural rubber latex (cis 1,4-polyisoprene), PVC (polyvinyl chloride), Nitrile (acrylonitrile and butadiene), Neoprene (chloroprene), plastic (polyethylene) or Tactylon (styrene-based copolymers).

In yet another embodiment, the at least one matrix is substantially biodegradable.

In yet another embodiment, the at least one matrix is self-degradable.

In yet another embodiment, the at least one matrix is substantially resorbable.

In yet another embodiment, the at least one matrix is substantially non-resorbable.

In yet another embodiment, the at least one matrix is in the form of a ribbon.

In yet another embodiment, the ribbon may be joined in the form of a loop such as to circumscribe the periphery of a tooth comprising the dental surface and the site.

In yet another embodiment, the at least one matrix is in the form of a cord.

In yet another embodiment, the cord may be joined in the form of a loop such as to circumscribe the periphery of a tooth comprising the dental surface and the site.

In yet another embodiment, the cord is made from catgut.

In yet another embodiment, the at least one matrix is in the form of a cervical corset.

In yet another embodiment, the corset may be fixed with respect to the dental surface and the site by means of one or more restraining straps adapted for securing the corset to a tooth comprising the dental surface.

In yet another embodiment, the straps circumscribe at least a portion of the tooth.

In yet another embodiment, the at least one matrix is in the form of an orthodontic interproximal “I” device.

In yet another embodiment, the “I” device may be fixed with respect to the dental surface and the site by means of an orthodontic arch wire previously secured in the intraoral cavity for securing the “I” device to a tooth comprising the dental surface.

In yet another embodiment, a system has been discovered for the controlled delivery of at least one material having a predetermined intra oral activity to a pit and fissures site, bucco-lingual interproximal surface or carious site, of at least one dental surface in an oral cavity, comprising at least one matrix containing the at least one material; the at least one matrix being adapted for affixing at the site for at least a predetermined time period correlated to the delivery of a predetermined portion of the at least one material to the site.

In yet another embodiment, the dental site may comprise enamel, cementum, dentine or any combination thereof.

In yet another embodiment, the matrix is in the form of an occlusal corset.

In yet another embodiment, the corset may be affixed with respect to the dental surface and the site by means of one or more restraining straps adapted for securing the corset to a tooth comprising the dental surface.

In yet another embodiment, the straps circumscribe at least a portion of the tooth.

In yet another embodiment, at least one the strap circumscribes at least a portion of an adjacent tooth.

In yet another embodiment, the matrix is in the form of at least part of a dental arch.

In yet another embodiment, the matrix is in the form of an entire dental arch.

In yet another embodiment, the matrix is in the form of an upper and lower dental arch.

In yet another embodiment, the arch comprises an outer layer that functions as an unresorbable or biodegradable/resorbable tray or shield.

In yet another embodiment, the system comprises more than one matrix, in a bi-layer, multi-layer, bi-location or multi-location form to deliver at least one agent in a single, bi, or multiphase controlled release pattern for appropriate or optimal delivery time, delivery stage, delivery manor, or delivery form.

In yet another embodiment, the system employs at least one matrix which keeps the active material inactive by chemical means or physical separation in order to allow at least one agent to be delivered at an appropriate or optimal time, stage, manner or form.

In yet another embodiment, the system employs at least one bi layer or multi layer matrix to initially keep at least one material separate thereby preventing the premature release of an agent or premature interactions.

In yet another embodiment, the at least one matrix comprise at least one adhesive surface or part thereof such as to enable the system to adhere or be fixed to a pit or fissure, smooth surface, carious, abraded or eroded surfaces or another dental site.

In yet another embodiment, the entire system or part thereof can be in a liquid, gel, foam, semi-liquid, colloidal or any form that can be painted onto a the dental surface where it solidifies with no intervention or with chemical or physical intervention such as light or heat application.

In yet another embodiment, unwanted or premature reactions are inhibited by use of chemical or physical means such as the use a varnishing, coating or encapsulation agent.

A method for the prevention and/or treatment of dental caries in a patient in need thereof has also been discovered, comprising applying at a site, such as a cervical root surface, interproximal surface, occlusal surface or carious lesion of the patient, wherein the at least one matrix is a fluoridation agent.

In one embodiment of the method, the material is selected from the group consisting of sodium fluoride, stannous fluoride, stannous hexafluorozirconate, calcium fluoride, difluorosilane, hydrogen fluoride, sodium monofluorophosphate, ytterbium trifluoride, sodium hexafluorosilicate, ammonium fluoride, acidulated phosphate fluoride, an amine fluoride, fluoroaluminosilicate glass and any mixture thereof.

In another embodiment, the method for the prevention and/or treatment of dental caries in a patient in need thereof, comprises applying at a site, such as a cervical root surface, interproximal surface, occlusal surface or carious lesion, of the patient, the at least one matrix, wherein the material is an amorphous mineral.

In yet another method, the material is selected from the group consisting of amorphous calcium phosphate, amorphous calcium phosphate fluoride, amorphous calcium carbonate phosphate, amorphous calcium carbonate phosphate fluoride, amorphous calcium fluoride and dicalcium phosphate dehydrate.

In yet another embodiment, the method for the prevention and/or treatment of dental caries in a patient in need thereof, comprises applying at a site, such as a cervical root surface, interproximal surface, occlusal surface or carious lesion, of the patient, the at least one matrix wherein the material is a crystalline mineral.

In yet another embodiment of the method, the material is selected from the group consisting of aragonite, brushite, calcite, dahltite, ferrhydrite, fluoroapatite, hydroxyapatite, lepidocrocite, magnetite, octacalcium phosphate, vaterite and whitlockite

In yet another embodiment, the method for the prevention and/or treatment of dental caries in a patient in need thereof comprises applying at a site, such as a cervical root surface, interproximal surface, occlusal surface or carious lesion, of the patient, the at least one matrixwherein the material is made of an organic material.

In yet another embodiment of the method, the material is selected from the group consisting of macromolecules such as acidic proteins, glycoproteins or sulfated polysaccharides, or smaller molecules such as polyaspartic or polyglutamic acid.

In yet another embodiment, the method for the prevention and/or treatment of dental caries in a patient in need thereof comprises applying at a site, such as a cervical root surface, interproximal surface, occlusal surface or carious lesion, of the patient, the at least one matrix wherein the material is an enhancing agent or further active agent.

In yet another embodiment of the method, the material is selected from the group consisting of calcium chloride, calcium bromide, calcium nitrate, calcium acetate, calcium gluconate, calcium benzoate, calcium glycerophosphate, calcium formate, calcium fumarate, calcium lactate, calcium butyrate, calcium isobutyrate, calcium maleate, calcium maleate, calcium propionate, calcium vaerate, alkali salts, ammonium salts of orthophosphoric acid such as potassium sodium or ammonium orthophosphate, monopotassium phosphate, dipotassium phosphate tripotassium phosphate, monosodium phosphate, disodium phosphate and trisodium phosphate.

In yet another embodiment, the method for the prevention and/or treatment of dental caries in a patient in need thereof comprises applying at a site, such as a cervical root surface, interproximal surface, occlusal surface or carious lesion, of the patient, the at least one matrix wherein the material is an acidifying, buffering or pH regulating agent.

In yet another embodiment of the method, the material is selected from the group consisting of acidulated phosphate fluoride, citric acid, sodium citrate, sodium bicarbonate, calcium carbonate, arginine and polyacrylic acid fully neutralized with alkalimetal ammonium or (alkylol) amine compound sodium polyacrylate.

In yet another embodiment, the method for the prevention and/or treatment of dental caries in a patient in need thereof comprises applying at a site, such as a cervical root surface, interproximal surface, occlusal surface or carious lesion, of the patient, the at least one matrix wherein the material is an antimicrobial agent.

In yet another embodiment of the method, the material is selected from the group consisting of stannous fluoride, alexidine, chlorhexidine digluconate, hexetidine, copper zinc citrate and stannous pyrophosphate, triclosan, cetylpyridinium chloride and halogenated bisphenolic compounds.

In yet another embodiment, the method for the prevention and/or treatment of dental caries in a patient in need thereof comprises applying at a site, such as a cervical root surface, interproximal surface, occlusal surface or carious lesion, of the patient, the at least one matrix wherein the material serves as a cleaning agent.

In yet another embodiment of the method, the material is selected from the group consisting of sodium alkyl sulfate, sodium lauryl sulfate, sodium coconut monoglyceride sulfonates, sodium lauryl sarcosinate, taurates, sodium lauryl sulfoacetate, sodium lauroyl isothionate, sodium laureth carboxylate, sodium dodecyl benzenesulfonate, poloxamers, polyoxyethylene sorbitan esters, fatty alcohol ethoxylates, polyethylene oxide, cocamidoppropyl betaine, sodium bicarbonate, monosodiumphosphate, sodium hydroxide, potassium hydroxide, sodium carbonate and imidazole.

In yet another embodiment, the method for the prevention and/or treatment of dental caries in a patient in need thereof comprises applying at a site, such as a cervical root surface, interproximal surface, occlusal surface or carious lesion, of the patient, the at least one matrix wherein the material serves as an effervescing agent.

In yet another embodiment of the method, the material uses a sodium bicarbonate/citric acid system.

In yet another embodiment, the method for the prevention and/or treatment of dental caries in a patient in need thereof comprises applying at a site, such as a cervical root surface, interproximal surface, occlusal surface or carious lesion, of the patient, the at least one matrix wherein the material serves as a tooth desensitizing agent.

In yet another embodiment of the method, the material is selected from the group consisting of fluorides, potassium citrate, potassium chloride, potassium tartrate, potassium bicarbonate, potassium oxalate and potassium nitrate.

In yet another embodiment, the method for the prevention and/or treatment of dental caries in a patient in need thereof comprises applying at a site, such as a cervical root surface, interproximal surface, occlusal surface or carious lesion, of the patient, the at least one matrix wherein the material serves as a tooth whitening or bleaching agent.

In yet another embodiment, the material is selected from the group consisting of hydrogen peroxide, carbamide peroxide metal chlorites, perborates, percarbonates, peroxyacids, persulfates, urea peroxide, calcium peroxide, calcium chlorite, barium chlorite, magnesium chlorite, lithium chlorite, sodium chlorite, potassium chlorite, hypochlorite, chlorine dioxide, sodium percarbonate, oxones, and protease.

In yet another embodiment, the at least one matrix comprises a synthetic polymer or a natural polymer which may be any one of polysaccharides, lipids, polyisoprene, gum, protein, or any mixture thereof.

In yet another embodiment, the at least one matrix comprises a synthetic polymer or a natural polymer which may be any one of polysaccharides, lipids, polyisoprene, gum, protein, or any mixture thereof.

In yet another embodiment, the natural polymer is a protein selected from denatured collagen, gelatin, denatured gelatin, chitosan silk or cellulose.

In yet another embodiment, the natural polymer is a protein selected from denatured collagen, gelatin, denatured gelatin, chitosan silk or cellulose.

In yet another embodiment, the polymer is cross-linked.

In yet another embodiment, the polymer is cross-linked or bound by at least one of glutaraldehyde, formaldehyde, glycol dimethacrylate, tannic acid, allyl methacrylate, nordihydroguaiaretic acid, rosemarinic acid.

In yet another embodiment, the at least one matrix further comprises any one of an enhancing agent for enhancing the application and release of the active material such as plasticizer, elasticizer, coloring agents, adhering agent, filler, softener, binder and preserving or sterilizing agent or any one of an auxiliary agent such as an antimicrobial agent, anti plaque agent, anti inflammatory agent, antioxidant, humoctant, nutrient analgesic or anaesthetic agent, anti calculus agent, cleaning agent, effervescent agent, tooth desensitizing agent, staining agent, hemostatic agent, whitening or bleaching agent, flavoring or sweetening agent, breath freshener, or sensate.

In yet another embodiment, the at least one matrix further comprises any one of an enhancing agent for enhancing the application and release of the active material such as plasticizer, elasticizer, coloring agents, adhering agent, filler, softener, binder and preserving or sterilizing agent or any one of an auxiliary agent such as an antimicrobial agent, anti plaque agent, anti inflammatory agent, antioxidant, humoctant, nutrient analgesic or anaesthetic agent, anti calculus agent, cleaning agent, effervescent agent, tooth desensitizing agent, staining agent, hemostatic agent, whitening or bleaching agent, flavoring or sweetening agent, breath freshener, or sensate.

In yet another embodiment, a support member has been discovered for fixing a polymeric matrix comprising a material having a predetermined intraoral activity to an interproximal site of at least one dental surface in an intraoral cavity, the support member comprising a peripheral frame portion surrounding a net portion, the frame portion being made from a resilient material capable of enabling the support member to be accommodated at the interproximal site such as to align the net portion therewith, and wherein the net portion is adapted for accommodating the matrix and for enabling the material to be delivered there from to the site.

In yet another embodiment, the frame member is in the form of a ring, wherein the member is attached to the inner concave surface of the ring.

In yet another embodiment, the frame member further comprises at least one niche for facilitating gripping of the the frame member to enabling affixing thereof at the interproximal site.

In yet another embodiment, the frame member further comprises at least one loop for facilitating gripping of the the frame member to enabling affixing thereof at the interproximal site.

In yet another embodiment, the support member is made from any suitable material including natural rubber latex (cis 1,4 polyisoprene), PVC (polyvinyl-chloride), Nitrile (acrylonitrile and butadiene), Neoprene (chloroprene), plastic (polyethylene) or Tactylon (styrene-based copolymers).

The above embodiment of the spherical or dumb-bell form includes a single, bi-layer, multi-layer, bi-location or multi-location system where in the latter formats an internal portion or layer may be in a liquid, semi-liquid, colloidal or gel form. After biodegradation, resorption or dissolution of the outer layer the liquid phase could be released and act locally and as an oral rinse within the mouth as a whole.

In yet another embodiment of the present invention, the system further comprises a suitable support member for fixing said matrix or matrices to said site, said support member comprising a peripheral frame portion surrounding a net portion, said frame portion being made from a resilient material capable of enabling the support member to be accommodated at said interproximal site such as to align said net portion therewith, and wherein said net portion is adapted for accommodating said matrix or matrices and for enabling said material or materials to be delivered therefrom to said site. The frame member may be in the form of a ring, wherein said member is attached to the inner concave surface of said ring. Optionally, the frame member further comprises at least one niche, and preferably two niches, for facilitating gripping of the said frame member to enabling affixing thereof at the interproximal site. Alternatively, the frame member may further comprise at least one loop, and preferably two, for facilitating gripping of the said frame member to enabling affixing thereof at the interproximal site. The support member is preferably made from any suitable material including natural rubber latex (cis 1,4-polyisoprene), PVC (polyvinyl chloride), Nitrile (acrylonitrile and butadiene), Neoprene (chloroprene), plastic (polyethylene) or Tactylon (styrene-based copolymers). In another embodiment, the matrix or matrices is are in the form of a ribbon, which may be joined in the form of a loop such as to circumscribe the periphery of a tooth comprising said dental surface and said site.

In another embodiment, the matrix or matrices is in the form of a cord, which may be joined in the form of a loop such as to circumscribe the periphery of a tooth comprising said dental surface and said site. The cord may be similar, physically and chemically, to catgut or made from any suitable material.

In another embodiment the matrix or matrices are in the form of a ribbon or cord which can be positioned for example on or in a pit or fissure or tooth-restoration interface of a said dental surface and said site to facilitate the controlled delivery of at least one material with or without chemical and or physical intervention such as laser application.

In another embodiment the matrix or matrices are in a form which fits over at least a portion of a dental arch for example thereby covering the occlusal, buccal and palatal/lingual tooth surfaces.

In another embodiment, the matrix or matrices is in the form of a cervical corset, which may be fixed with respect to said dental surface and said site by means of one or more restraining straps adapted for securing said corset to a tooth comprising said dental surface. The straps typically circumscribe at least a portion of said crown end and/or root of said tooth.

In another embodiment, the matrix or matrices is in the form of an orthodontic interproximal “I” device, which may be fixed with respect to said dental surface and said site by means of an orthodontic arch wire previously secured in the intraoral cavity for securing said “I” device to a tooth comprising said dental surface.

In another embodiment the matrix or matrices is in any form such as those mentioned above to be positioned at a said dental surface such as root dentin, occlusally, cervically, interproximally or within a carious lesion to provide a at least one material for a change induced by a heat or light (for example laser; see Westerman G. H. et al, J. Clin. Laser Med. Surg. 17 63-68, 1999) source to thereby alter said dental surface in order to facilitate prevention, cosmetic changes, desensitization healing or restoration of said lesion.

In a further aspect, the present invention also relates to a system for the controlled delivery of a material or materials having a predetermined intra oral activity to an occlusal site of at least one dental surface in an oral cavity, comprising a matrix or matrices containing said material or materials; said matrix or matrices being adapted for affixing at said occlusal site for at least a predetermined time period correlated to the delivery of a predetermined portion of said material or materials to said site. In one embodiment, the matrix or matrices are in the form of an occlusal corset, which may be affixed with respect to said dental surface and said site by means of one or more restraining straps adapted for securing said corset to a tooth comprising said dental surface. Preferably, the straps circumscribe at least a portion of said tooth, and at least one said strap circumscribes at least a portion of an adjacent tooth.

For all embodiments, the active material or materials may be, for example, any one or more of an inorganic or organic fluoride-containing chemical agent known or to be developed.

The embodiments of this invention are not limited to fluoride agents but may contain other remineralizing or mineralizing agents, and combinations thereof.

Another embodiment is to optimize the fluoride dose and chemical type and the efficiency of fluoride availability for remineralization or mineralization.

The matrix or matrices may comprise a synthetic polymer, a natural polymer or derived thereof which may be at least one of polysaccharides, lipids, polyisoprene, gum and proteins, or any mixture thereof. The natural polymer may be a protein selected from collagen, silk, elastin or a derived natural product such as gelatin. Preferably, the polymer is cross-linked, typically by at least one of allyl methacrylate, 2,3, dihydroxybenzaldehyde, 3,4, dihydroxybenzaldehyde, formaldehyde, glycol dimethacrylate, glutaraldehyde, nordihydroguaiaretic acid, rosemarinic acid, and/or tannic acid.

The present invention also relates to a support member for fixing at least one polymeric matrix comprising at least one material having a predetermined intraoral activity to an interproximal site of at least one dental surface in an intraoral cavity, said support member comprising a peripheral frame portion surrounding a net portion, said frame portion being made from a resilient material capable of enabling the support member to be accommodated at said interproximal site such as to align said net portion therewith, and wherein said net portion is adapted for accommodating said matrix or matrices and for enabling said material or materials to be delivered therefrom to said site. In one embodiment, the frame member is in the form of a ring, wherein said member is attached to the inner concave surface of said ring. The frame member optionally further comprises at least one niche for facilitating gripping of the said frame member to enabling affixing thereof at the interproximal site. Alternatively, the frame member further comprises at least one loop for facilitating gripping of the said frame member to enabling affixing thereof at the interproximal site. Typically, the support member is made from any suitable material including natural rubber latex (cis 1,4 polyisoprene), PVC (polyvinyl-chloride), Nitrile (acrylonitrile and butadiene), Neoprene (chloroprene), plastic (polyethylene) or Tactylon (styrene-based copolymers).

The matrix or matrices optionally further comprises an auxiliary agent which may be any one of an enhancing agent for enhancing the release of the active material or materials, or an agent to enhance physical properties of the device such as softness, color elasticity and strength, chemical properties such as self-life, anti-microbial activity, adhesive properties, coloring and agents with other functions such as a staining agent, anti-inflammatory agents cleansing agent whitening or bleaching agent, desensitizing agent, haemostatic agent, vasoconstrictor and a preserving or sterilizing agent.

### BRIEF DESCRIPTION OF THE FIGURES

FIGS. 1(a) and 1(b) illustrate, in side view, a labial and buccal portion of the anterior teeth and the posterior teeth, respectively, illustrating the three major tooth zones that are particularly susceptible to dental caries.

FIGS. 2(a) to 2(d) illustrate, in front view, alternative configurations according to a first embodiment of the present invention; FIGS. 2(e) and 2(f) illustrate, in side view, a portion of the anterior teeth and the posterior teeth, respectively, illustrating the configurations of FIGS. 2(a) to 2(d) fixed in situ with respect to various interproximal zones. FIG. 2(g) illustrates a transverse cross-sectional view of the embodiment of FIG. 2(a) taken along x-x.

FIGS. 3(a) to 3(d) and 3(h) illustrate, in front view, alternative configurations according to a second embodiment of the present invention; FIGS. 3(e) and 3(f) illustrate, in side view, a portion of the anterior teeth and the posterior teeth, respectively, illustrating the configurations of FIGS. 3(a), 3(b) and the configuration of FIG. 3(c), respectively, fixed in situ with respect to various interproximal zones. FIG. 3(g) illustrates a transverse cross-sectional view of the embodiment of FIG. 3(a) taken along y-y.

FIGS. 4(a) to 4(c) illustrate, in front view, alternative configurations according to a third embodiment of the present invention. FIGS. 4(d) and 4(e) illustrate, in side view, a portion of the anterior teeth and the posterior teeth, respectively, illustrating the configurations of FIGS. 4(a), 4(b) and of FIG. 4(c), respectively, fixed in situ with respect to various interproximal zones. FIG. 4(f) illustrates the configuration of a soft interproximal sphere and FIG. 4(g) is a transverse cross section view of the embodiment of FIG. 4(f), taken along z-z showing the two layers of the said embodiment which is also shown in side view in situ between posterior teeth (47), (FIG. 4h). These units can be linked for example by a strand or ribbon to facilitate placement of the system at least at one dental site, as shown in FIG. 4(h). The strand or ribbon or band can be the identical in composition as the unit or differ. For example it can resorb faster. Obviously this embodiment can be a single phase uniform system or a multi phase system and one or more of the internal phase or phases of a bi or multi layer system respectively can be in a liquid form. Further such as system does not have to be limited to a physical interproximal ball but can be larger or in different forms such as a flat rectangular form. These systems can thereby function as mouth rinses thus simplifying dispensing and use of an oral rinse. The delivery system of mouth rinses negates the cumbersome use of traditional oral rinses which necessitate storing and dispensing from plastic bottles. The outer layer once biodegraded, dissolved or absorbed then allow the inner agent or agents to be released.

FIGS. 5(a) and 5(b) illustrate, in perspective view, alternative configurations according to a fourth embodiment of the present invention; FIGS. 5(c) and 5(d) illustrate, in side view, a portion of the anterior teeth and the posterior teeth, respectively, illustrating the configurations of FIG. 5(a) and FIG. 5(b), fixed in situ with respect to various cervical and interproximal zones.

FIGS. 6(a) and 6(b) illustrate, in perspective view, alternative configurations according to a fifth embodiment of the present invention; FIG. 6(c) illustrates, in side view, a portion of the anterior teeth illustrating the configuration of FIG. 6(a) fixed in situ with respect to various cervical and interproximal zones. It is possible to have a single matrix where the portions 60 and 62 are identical, or the matrix can comprise an apical section 62 which differs from the coronal section 60. These advantages are discussed in more detail below. FIG. 6(d) illustrates, in perspective view, a portion of the posterior teeth, illustrating the configuration of FIG. 6(b) fixed in situ with respect to various occlusial and interproximal zones. FIG. 6(e) illustrates, in perspective view a portion of the posterior teeth, illustrating the configuration of 60, and 62 of FIG. 6(b) placed on occlusal surfaces.

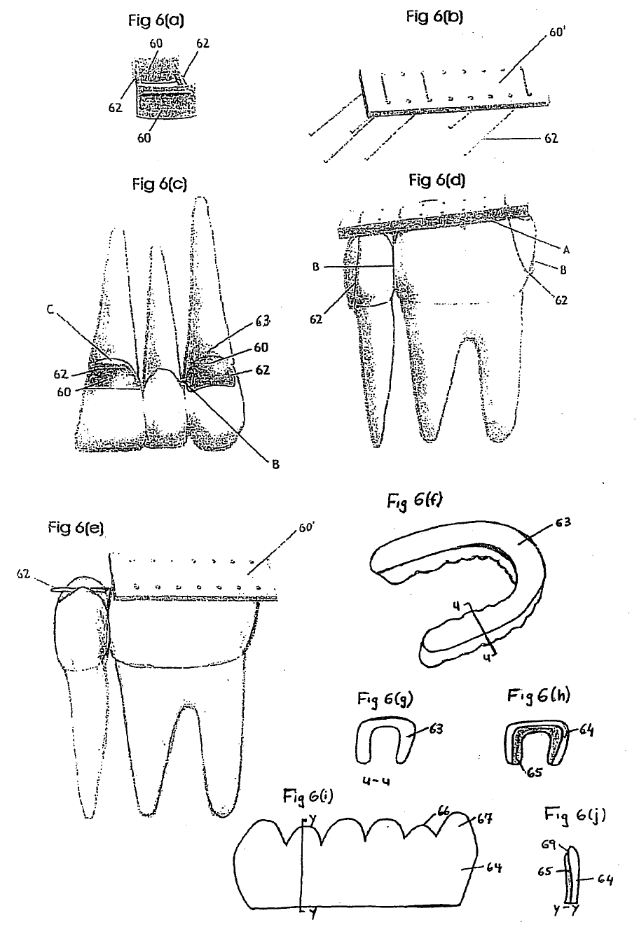

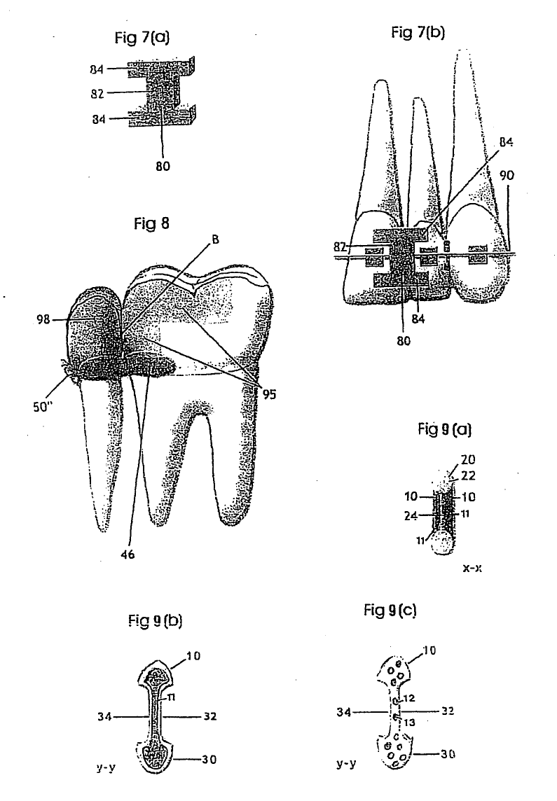

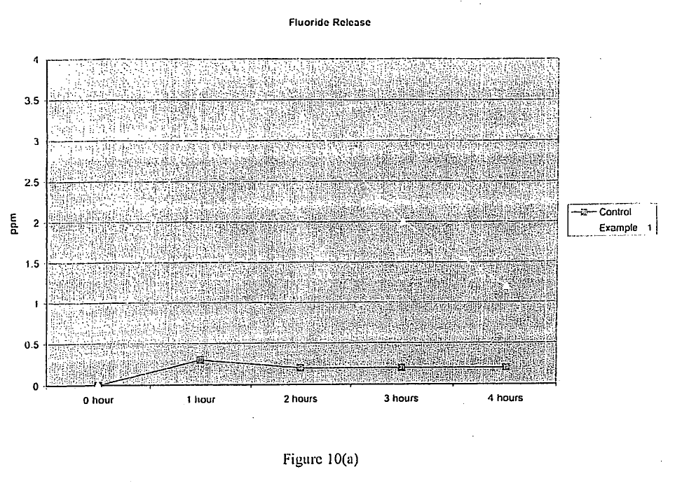

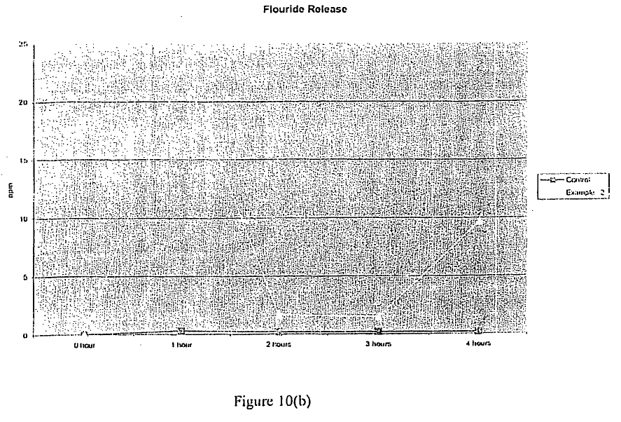

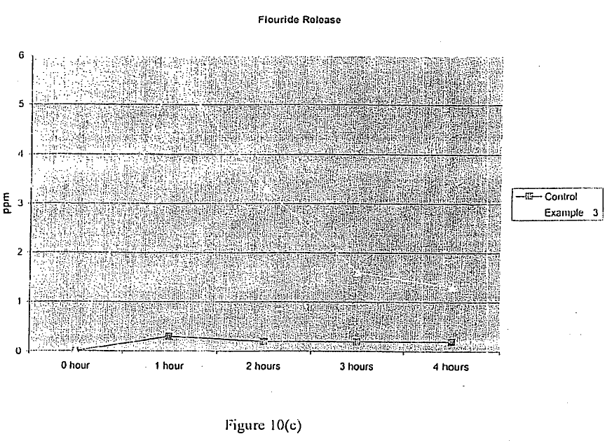

The invention includes a system which conforms at least to part of a dental arch and which would be for example a single system which in shape is a combination of 6(c) and 6(d) with or without the holes or strands. For example this system could cover the occlusal, buccal, palatal/lingual and portions of the interproximal sites. The system could cover part of a dental arch, and entire dental arch 63 (FIG. 6(f)) and/or the upper and lower dental arch. This single matrix system could also be designed to provide a 1-4 minute fluoride tray application within a standard tray or the matrix itself could also function as the tray (FIG. 6g). The system could comprise more than one matrix for example two layers whereby the outer layer (64) could function as unresorbable or a resorbable/ biodegradable tray and the inner biodegradable/resorbable layer (65) provides the active agent or agents FIG. 6(h)). The system may cover the anterior (buccal or labial) dental arch as a single system which is shaped like 6(c) at the apical portion of the crown, and extend to the incisal edge of the tooth, covering the entire anterior surface of the crown, or it may extend to the posterior (palatal or lingual) portion of the crown. The system also may not be limited to covering one tooth, but may cover more than one tooth, part of a dental arch or the entire arch on the labial/buccal and palatal/lingual portion (FIG. 6(f)), or only on one surface (FIG. 6(i)). The region close to the gingival may be shaped or scalloped to conform with the anatomy of the gingiva (66) and tooth (67). The system may comprise more than one matrix, for example, two layers, whereby the outer layer (64) could act as a shield to protect the soft tissue (e.g. lips) from the inner layer which provides the active agent or agents (65).

FIG. 7(a) illustrates, in front view, a sixth embodiment of the present invention; FIG. 7(b) illustrates, in side view, a portion of the anterior teeth illustrating the embodiment of FIG. 7(a) fixed in situ with respect to a labial tooth surface.

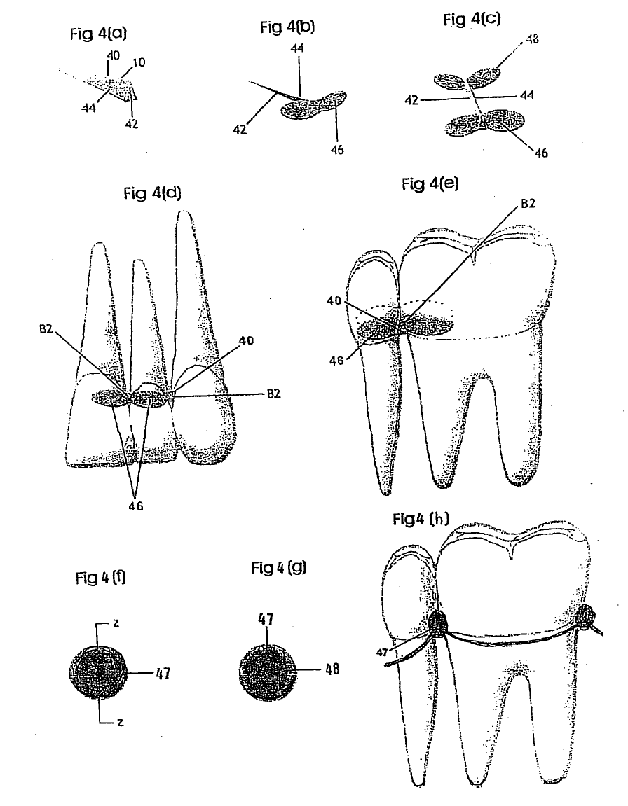

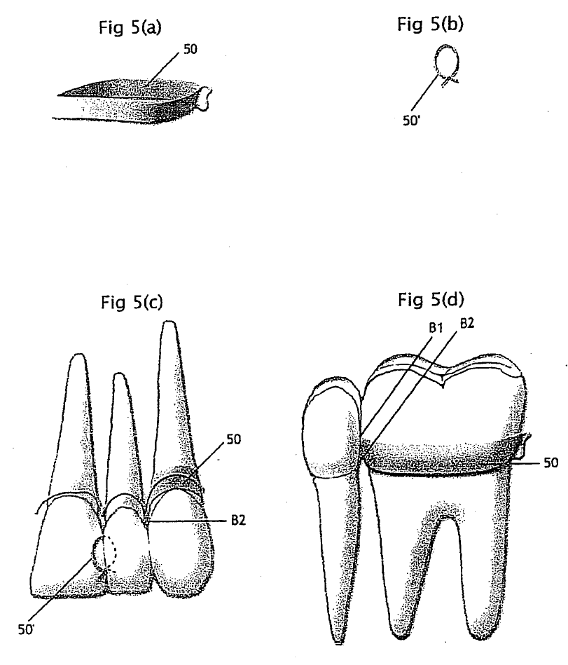

FIG. 8 illustrates, in side view, a portion of the posterior teeth, illustrating the configurations of FIG. 4(b) and FIG. 5(b) fixed in situ with respect to various interproximal and cervical zones.

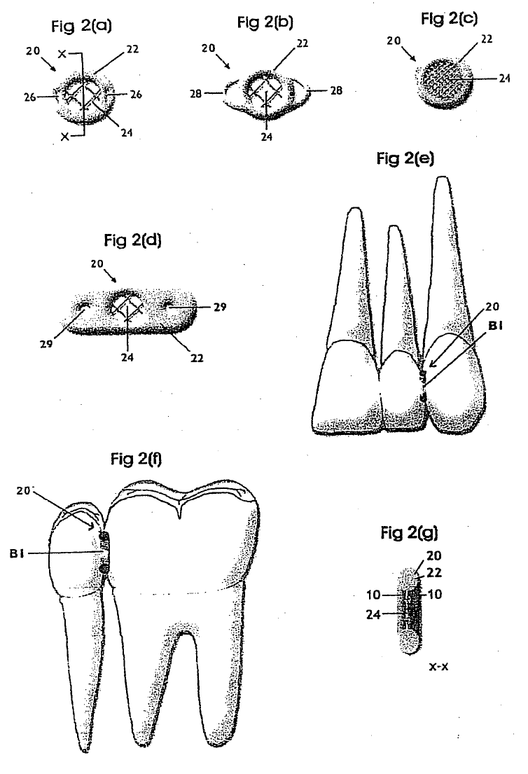

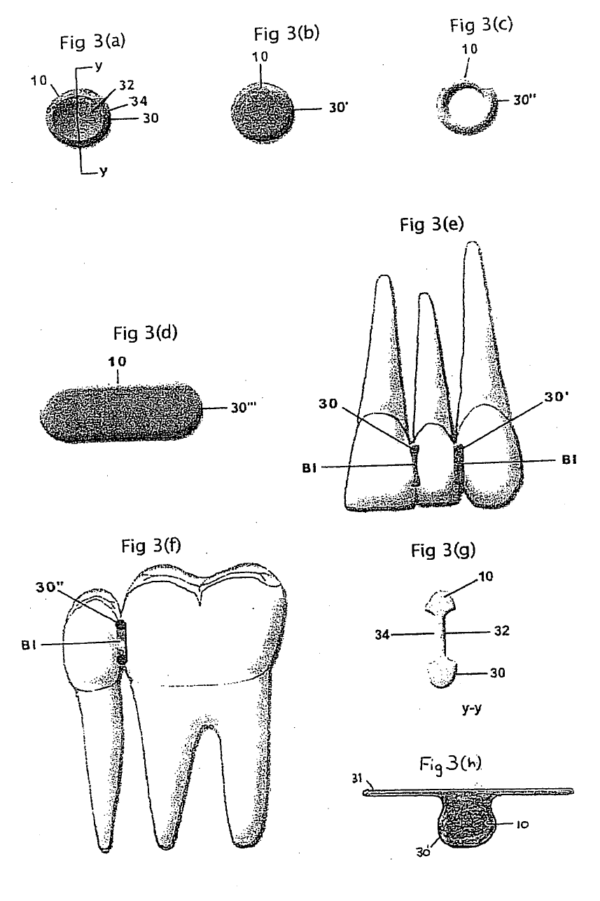

FIG. 9(a) illustrates a bi layer system similar to the transverse cross-sectional view of the embodiment of FIG. 2(a) taken along x-x. FIG. 9(b) illustrates a bi layer system similar to the transverse cross-sectional view of the embodiment of FIG. 3(a) taken along y-y. FIG. 9(c) illustrates a multilocated system similar to the transverse cross-sectional view of the embodiment of FIG. 3(a) taken along y-y.

FIGS. 10(a) to 10(c) show graphically the results obtained from experimentation, as described in Examples 1-3 below.

### DETAILED DESCRIPTION OF THE PREFERRED EMBODIMENTS

The present invention is defined by the claims, the contents of which are to be read as included within the disclosure of the specification, and will now be described by way of example with reference to the accompanying Figures.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The present invention relates to a system for the controlled or sustained delivery of a material or materials having a predetermined intra-oral activity to a dental site of the oral cavity, typically on tooth surfaces or carious lesions, and in particular to contact points/areas of an interproximal site of at least one dental surface of the oral cavity, the system comprising a matrix or matrices containing said material or materials. The matrix or matrices is adapted for the controlled or sustained release of the active material or materials, and is further adapted for fixation at the dental site, and in particular the interproximal site, for at least a predetermined time period that is correlated to the delivery of a predetermined portion of said material or materials to said site. This time period typically depends on the nature of the active material or materials and on the subject being treated, and may comprise a few seconds while a heat or light source such as a laser is administered to about four hours during interproximal caries prevention or treatment or indeed even about four days, for example, when fluoridizing an interproximal site during tooth separation according to the present invention. It is to be appreciated that a major factor in establishing the rate of release of the active material or materials is the structure of the polymeric matrix or matrices as a single uniform unit, multi-layer or a multi-location form. Thus, desired rates of release may be achieved by employing specific polymers, which are preferably cross-linked to a degree affording the desired rate of release. Matrices that are highly cross-linked would release the active material or materials more slowly, and vice versa. The man of skill in the art of pharmacy and delivery system is familiar with such considerations, which are described in many articles and textbooks, e.g., Remington's Pharmaceutical Sciences, Gennaro A. R. ed., Mack Publishing Company, Easton Pa., 1990, which is fully incorporated herein by reference.

The release of an active agent or agents can be varied within a single matrix or by utilizing a combination of more than one matrix. There are many examples of means for varying release patterns from a single matrix. Examples include different types and degrees of cross-linkage and different additives (such as antimicrobial agents, preservatives, sterilizing agents and enzyme inhibitors) which influence the biodegragation. Furthermore, the release of even a single agent can vary by the manner it is bound in a matrix. For example, sodium fluoride can be released from a single matrix in a biphasic manner where the initial release is of loosely bound sodium fluoride and the next release is of more firmly bound sodium fluoride (see Example 2). Different patterns can also be obtained by using different types of fluoride, for example sodium fluoride, acidulated phosphate fluoride and an amino fluoride, which differ chemically and in molecular size. Another facet is that the matrix can create a microenvironment which excludes some salivary products such as large proteins that inhibit mineralization, and others which include mineralization such as calcium phosphate and arginine. The biphasic pattern of sodium fluoride release (Example 2) allows an initial burst of fluoride ions to exchange with hydroxyapatite OH^{—} groups, then the lapse of fluoride release allows the crystals to grow by providing calcium and phosphate from the matrix, either added as agents to the matrix or absorbed by the matrix from the saliva. The final release then favors the deposition of calcium fluoride globules which are long term pH sensitive fluoride reservoirs.

Another approach of varying release patterns is the use of more than one matrix either as separate layers or multilocated systems (see FIG. 9). Besides causing different release patterns, the use of more than one matrix can keep different agents apart to in situ placement . Each matrix could be loaded with the same or different agent/s that could be released at different rates and/or stages by utilizing intrinsically different matrices at the chemical level or/and physical parameters. For example, the outer layer of a bilayer sphere would first be exposed to the saliva and release, for example, an effervescent cleaning system which loosens and dislodges interproximal plaque and debris and then the inner layer releases, for example, fluoride ions. Another example is that the device can comprise of a coronal and an apical region where the coronal region contains an agent or agents more effective on enamel and the apical region contains an agent or agents more effective on cementum and dentin (see FIG. 6(c)). An example of a multilayered multi-phase release system is one designed to mimic chiton radula formation which could be used to favorably alter tooth surfaces.

These matrices can comprise a single unit which was affixed one upon the other either by physical pressure or chemical bonding. They can also be formed by plating the first layer and then the same layer is plated over the dried first layer.

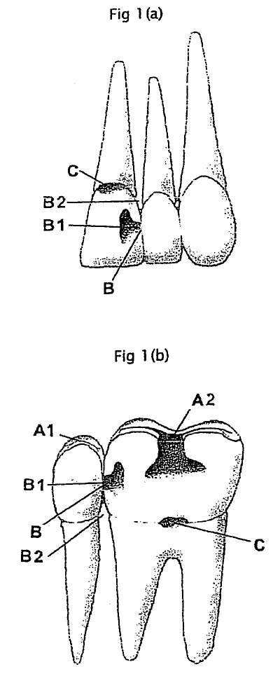

Referring to FIG. 1(a) and FIG. 1(b), the interproximal site (B) comprises a point/area (B1) of contact between a dental surface of interest, i.e., wherein it is desired to deliver the active material or materials, and an adjacent dental surface. The interproximal site (B) also comprises a space (B2) where the adjacent teeth do not touch. According to the present invention, said active material or materials may be delivered to the point/area of contact (B1) and/or space (B2) where the teeth do not make direct contact, and to either one or both of the adjacent teeth. The delivery may be sustained or instantaneous when complimented with a light or heat source. The agent may be a mineralizing or remineralizing agent and/or a cleansing agent for the said site.

The occlusal site includes natural pits and fissures (A1) and carious lesions (A2) at said sites. According to the present invention the said system may be applied with or without other chemical and/or physical intervention such as cavity or fissure extension, heat or light applications. An example of a light source is a laser. The cervical zones (C) can involve only root dentin cementum and be exclusively limited to enamel or a combination. Root caries can be similar to some interproximal caries when they are located in the B2 zone.

In a first embodiment of the present invention, and referring to FIGS. 2(a) to 2(g), the delivery system comprises a polymeric matrix (10) containing the active material or materials, and a suitable carrier or support member (20) for fixing said matrix (10) to the desired interproximal site, typically the point/area of contact (B1) thereof. Said support member (20) comprises a peripheral frame (22) surrounding a net portion (24). The frame portion (22) is typically made from a resilient material capable of enabling the support member (20) to be accommodated at the corresponding area of contact (B1) of the interproximal site (B) such as to align said net portion (24) therewith, as illustrated in FIGS. 2(e) and 2(f). The net portion (24) is adapted for accommodating and retaining the said matrix (10) and for enabling the active material or materials contained therein to be delivered from the matrix (10) to the site (B). In the FIGS. 2(e) to 2(g) the matrix (10) is presented as a single uniform matrix (10) but the invention includes other possibilities such as bi or multi-layer or bi or multi-location forms an example of which is illustrated in FIG. 9(a). Furthermore, the net portion (24) also facilitates the positioning of the matrix (10) at the interproximal site. Thus, the net portion (24) acts as a retentive, receiving vessel or seat for the matrix (10) affixing the matrix (10) to the support member (20). At the same time the plurality of apertures or orifices comprised in the net portion (24) also enables the active material or materials to be delivered to both adjacent teeth at the area of contact (B1), as illustrated in FIGS. 2(a) to 2(d).

Referring to FIG. 2(a), in a first configuration of the support member (20), the frame member (22) is substantially ring-like or annular, wherein said net member (24) is attached to the inner cylindrical or concave surface of the ring. In the first configuration, the frame member (22) also comprises a pair of diametrically opposed niches (26) for the purpose of positioning the support member (20) in between the teeth. This is accomplished by placing the beaks of a rubber dam pliers, orthodontic pliers, a custom designed pliers, dental floss, or any other apparatus which stretches the member (20) or do not stretch the member (20) such as traditional wedges, Magege interdental star wedges, or tooth separators such as Elliot separators or TruFlex™ Nickel Titanium separators, in order to maneuver it interproximally.

A second configuration of the first embodiment, illustrated in FIG. 2(b), comprises the same elements as described hereinbefore with respect to the first configuration, mutatis mutandis, with the exception that instead of the niches (26), the frame member (22) also comprises a pair of diametrically opposed loops (28) extending from the outer rim of the frame member (22) for the purpose of placing the support member (20) in-between adjacent teeth, as for the first configuration. However, the loops (28) have an additional advantage in that wider beaks may be used, and in that they are also enable the support member (20) to be stretched by hand.

A third configuration of the first embodiment, illustrated in FIG. 2(c), comprises the same elements as described hereinbefore with respect to the first configuration, mutatis mutandis, with the exception that the net member comprises a smaller mesh than in the first configuration, and furthermore lacks the said niches (26).

In a fourth configuration of the first embodiment, illustrated in FIG. 2(d), the frame member is in the form of a strip having a centrally disposed circular aperture, wherein said net member (24) is attached to the inner cylindrical or concave surface of the aperture. In the fourth configuration, the frame member (22) also comprises a pair of diametrically opposed niches (29) aligned along the length of the strip for the purpose of placing the support member (20) in-between adjacent teeth, as for the first configuration. However, the additional length afforded by the strip facilitate placement by hand.

In the first embodiment of the present invention, the frame member (22) is typically integral with the net member (24) and may be made as a molded item from any suitable materials that facilitate placement of the active material or materials within the support member (20) and to enable the support member (20) to be easily slipped in between the teeth. Such suitable materials may include natural materials such as, for example, natural rubber latex (cis 1,4 polyisoprene), or synthetic materials such as, for example, PVC (polyvinyl-chloride), Nitrile (acrylonitrile and butadiene), Neoprene (chloroprene), plastic (polyethylene) or Tactylon (styrene-based copolymers made by the Safeskin Corporation). Placement of the support member (20) including the matrix (10) may be accomplished by stretching the support member (20) by hand, with floss, with a rubber dam pliers, orthodontic pliers, or a custom designed pliers with optimally shaped beaks or the placement may be accomplished with suitable traditional wedges, Magege interdental star wedges or tooth separators such as Elliot separators or TruFlex™ Nickel Titanium separators. As the support member (20) typically needs to be stretched into about 4-5 times its unstressed diameter, the physical properties of the active material and in particular the matrix (10) will have to include suitable elastic properties which match that of the elastic support member (20). Other shapes, for example oval or square, are also within the scope of the invention. The net portion (24) can vary in the mesh open area provided thereby, and may be made from the same material as the frame member (22) or from a different material thereto, for example, such as nets or fibrefilms used in triple impression trays.

The first and third configurations may comprise an external diameter ranging, typically, between 3 mm and 9 mm, particularly between about 3.8 mm and 4.6 mm. The external dimension of the second configuration may be similar, not including the loops (28), and so too the width of the fourth configuration. When orthodontic separation of teeth is to be instituted, the first embodiment may thus be conveniently used to provide preventive fluoridization of the interproximal regions.

The first embodiment may also be used to remineralize demineralized interproximal regions of teeth which have been detected visually or radiologically. Further, a programmed prevention technique can be used to strategically fluoridate teeth as they erupt.

According to the first embodiment of the present invention, it may be necessary to separate the teeth using orthodontic techniques, in order to position the support member and matrix (10) therein. However, such a separation is typically much less than in regular orthodontic practice.

Thus, in the first embodiment of the present invention, the support members are formed such as to embody the appropriate shape to contact the tooth surface and retain its position as well as to facilitate ease of application in the targeted areas by embodying adequate elasticity and toughness for the contact areas or points. Hence a wide range of shapes can be manufactured, for example in pre-designed moulds (e.g. of plastics, metal or rubber) where the chemical components (including the matrix (10) and at least one active material) are placed or injected and set, using chemical and/or physical means (e.g. chemical interactions, concentration changes, pressure, temperature and/or irradiation). Another example is the production of sheets of a suitable material from which desired shapes of the support member can be cut or punched out. The support members, for example as exemplified in FIGS. 2(a) to 2(e), may be produced in the form of individual members, or alternatively attached to a “mother” branch or branches. These branches comprising the support members can then be treated with the active material or materials, such as for example remineralizing agent, itself by flowing the wet or liquid material into the retention portions of these members and then dried or set. In accordance with further embodiment of the present invention, any alternative suitable technique may be used to produce the device or the material or materials.

Alternatively, the support member of the first embodiment may be adapted for carrying the matrix (10) at a micro-scale. In such a configuration, the support member is made from a suitable porous material, having pores, tubules or any other micro-scale structures for containing the matrix (10), rather than the net portion for accommodating the matrix (10). The active material or materials is then released from the pores and to the interproximal site. Such pores/tubules may be provided by laser drilling techniques applied to a support member made from regular latex rubber. Alternatively, during the manufacturing process of the support member, chemical, air or any suitable gas may be introduced to the molten latex rubber at suitable stage(s)/temperature/pressure to form a cellular or porous structure therein when set.

While the support member of the first embodiment is preferably made from non-resorbable materials such as latex rubber, for example, the support member may nevertheless be made, alternatively, from resorbable and/or biodegradable materials that are typically more resilient or durable than the material of the matrix (10) itself. For example, the support member may be made from the same basic material as the matrix (10), but cross-linked by cross-linking agents that are stronger, or more concentrated, or agents other than used for cross-linking the matrix (10).

In a second and third embodiments according to the present invention, and in fact also in the first embodiment, the matrix (10) is a polymeric matrix (10), which may be biodegradable, resorbent or non-resorbent, and comprises a hydrophilic polymer such as to enable the matrix (10) to be retained in place, optimizing surface contact by swelling in situ by the hydration thereof in the oral cavity after accommodation at said interproximal site. Furthermore, the polymeric matrix (10) has a three dimensional form having an external surface, wherein at least a portion of said external surface is adapted for contact with one of the two adjacent teeth, at the contact point/area (B1) and/or the non-contact space (B2) of the interproximal site (B), such as to deliver said at least one material to said site. Alternatively, the three dimensional form of the matrix (10) has two distinct surfaces, a first external surface and a second external surface. In the latter case, at least a portion of said first external surface is adapted for contact with dental surface of one tooth at the area of contact (B1) and/or the space (B2) of said interproximal site (B), and at least a portion of said second external surface is adapted for contact with the dental surface of the adjacent tooth at the area of contact (B1) and/or the space (B2), such as to deliver the active material or materials to both adjacent teeth at the interproximal site (B). As with the first embodiment, the adjacent teeth are actually separated at area of contact (B1) when the delivery system is fixed in place.

Thus, in the second embodiment, and referring to FIG. 3(a) the polymeric matrix (10) is particularly adapted for providing the active material or materials to the contact area (B1).

In a first configuration of the second embodiment, and referring to FIGS. 3(a) and 3(g), the polymeric matrix (10) is in the form of a disc (30) having opposed first and second external substantially concave surfaces, (32) and (34) respectively, for contact with at least the contact area (B1) of said dental surfaces of the adjacent teeth at the of the interproximal site (B) such as to deliver said at least one material to said site.

In a second configuration of the second embodiment, and referring to FIG. 3(b), the polymeric matrix (10) is in the form of a disc (30′) having opposed first and second external substantially flat surfaces, for contact with at least the contact area (B1) of said dental surfaces of the adjacent teeth at the of the interproximal site (B) such as to deliver said material or materials to said site.

In FIGS. 3(a) to 3(h) the matrix (10) is presented as a single uniform matrix (10) but the invention includes other possibilities such as a bi or multi layered or bi or multi-located form some of which are illustrated in FIGS. 9(b) and 9(c).

In a third configuration of the second embodiment, and referring to FIG. 3(c), the polymeric matrix (10) is in the form of a ring (30′) having opposed first and second external substantially annular surfaces, for contact with at least the contact area (B1) of said dental surfaces of the adjacent teeth at the of the interproximal site (B) such as to deliver said at least one material to said site.

In a fourth configuration of the second embodiment, and referring to FIG. 3(d), the polymeric matrix (10) is in the form of a longitudinal strip or pellet (30′″) having opposed first and second external substantially oval surfaces, for contact with at least the contact area (B1) of said dental surfaces of the adjacent teeth at the of the interproximal site (B) such as to deliver said at least one material to said site. This elongated configuration facilitates stretching by hand for positioning of the matrix (10) interproximally.

In a fifth configuration of the second embodiment, and referring to FIG. 3(h), the polymeric matrix (10) is in the form of a disc (30′) similar to that of FIG. 3(b), and is attached to a cord (31) to facilitate placement at an interproximal site. The cord (31) can be made of the same polymeric material as the matrix (10) or a different material. Alternatively, the matrix (10) may be in the form of the embodiment of FIG. 3(a). Following interproximal insertion of the disc (30′), the cord (31) may be removed by chemical or mechanical means.

FIGS. 3(a)-(d) and (h), can also include properties inherent in FIGS. 2(a)-2(d) as described above, mutatis mutandis, namely that the matrix (10) may be elastic enough to separate teeth and also release active agents. Additionally, the discs of FIGS. 3(a)-3(d) and 3(h) may be adapted for carrying the matrix (10) at a micro-scale, as in the first embodiment as described above, mutatis mutandis. Namely, wherein the disc is made from a suitable porous material, having pores, tubules or any other micro-scale structures for containing the matrix (10). The active material or materials is then released from the pores and to the interproximal site. Such pores/tubules may be provided by laser drilling techniques applied to a support member made from regular latex rubber. Alternatively, during the manufacturing process of the support member, air or any suitable gas may be introduced to the molten latex rubber at suitable temperature/pressure to form a cellular or porous structure therein when set.

Where necessary, a dental practitioner may pry adjacent teeth apart using, for example, a wooden device (e.g. a traditional wedge or a metal device (e.g. an Elliot Separator or a TruFlex™ Nickel Titanium separator) or a plastic device, (e.g. Magege Interdental star wedge), in order to facilitate placement of the system between them. Thereafter, the wedge or separating device is removed. Thus, in the second embodiment of the present invention, the polymeric matrix (10) is anatomically shaped to fit any tooth surface, for example buccal surfaces, fissures, carious lesions and the interproximal anatomy of the teeth (in order to reach the contact points or areas), for the slow release of the active material or materials, typically fluoride and/or other agents, or for targeted heat or light applications such as lasers which induce advantageous chemical changes due to the presence of the agent or agents held in position by the system of this invention as illustrated in FIGS. 3(e) and 3(f).

In the first, second, third and fourth configurations of the second embodiment, the external diameter may range, typically, between 3 mm and 9 mm, particularly between about 3.8 mm and 4.6 mm. Also, other shapes, for example oval or square, are also within the scope of the invention.

In the third embodiment, and referring to FIGS. 4(a) to 4(e) the polymeric matrix (10) is particularly adapted for providing the active material or materials to the space (B2). In a first configuration of the third embodiment, and referring to FIG. 4(a), the polymeric matrix (10) is in the form of a wedge (40) having first and second external substantially longitudinal surfaces, (42) and (44) respectively, for contact with at least the facing dental surface of the adjacent teeth at the interproximal space (B2) at the of the interproximal site (B) such as to deliver said at least one material to said site, close to the gingiva and towards the contact point/area, as illustrated in FIG. 4(d). In these FIGS. 4(a) to 4(e) the matrix (10) is presented as a single uniform matrix (10) but the invention includes other possibilities such as a bi or multi layered or bi or multi-located form similar to those illustrated in FIGS. 9(b) and 9(c) and 4(g).

In a second configuration of the third embodiment, and referring to FIG. 4(b), the polymeric matrix (10) is in the form of a wedge (40) having a one pair of winged members (46) at one longitudinal end thereof. Alternatively, the wedge (40) may comprise a winged member (46) at one longitudinal end thereof directed towards only one of the two adjacent teeth. As with the first configuration, the wedge (40) has first and second external substantially longitudinal surfaces, (42) and (44) respectively, for contact with at least the facing dental surface of the adjacent teeth at the space (B2) at the of the interproximal site (B) such as to deliver said at least one material to said site. Each of the winged members also have suitable contact surfaces which wrap around and deliver a portion of said at least one material to a portion of externally facing dental surfaces of the of the cervical regions of the two adjacent teeth, mesial and distal to said interproximal site. The winged members may optionally be long enough to wrap around each corresponding tooth and into the next interproximal site, as illustrated in FIGS. 4(d) and 4(e).

A third configuration of the third embodiment, and referring to FIG. 4(c) comprises all the elements described for the second configuration of the third embodiment, mutatis mutandis. Furthermore, the matrix (10) in the third configuration of the third embodiment also comprises a second pair of winged members (48) at the other longitudinal end of the wedge (40). Alternatively, the wedge (40) may comprise a winged member (46) at the second longitudinal end thereof directed towards only one of the two adjacent teeth. Each of the winged members (48) also have suitable contact surfaces for wrapping around and delivering a portion of said at least one material to a portion of the internally facing dental surfaces of the cervical regions of the two adjacent teeth, proximal and distal to said interproximal site. Thus, the third configuration of the third embodiments is in the form of an interproximal device which has its arms, i.e. the winged elements, folded to slip between the teeth before the arms are allowed to “spring” into the original position.

A fourth configuration of the third embodiment and referring to FIG. 4(f) comprises a soft hydrophilic dumb-bell or sphere which can comprise a single or a bi-layered system (47 and 48) as shown in FIG. 4(g) a multilayer or a multi-location system. The system is designed for easy insertion into an interproximal space below the contact area or point, as illustrated in FIG. 4(h). An internal layer may be in a liquid form which can also function as an easily dispensed oral rinse and the system need not be limited to interproximal placement.

An optional system can include strands linking one or more units for example two or more spheres. An example is 7 sphere units to facilitate placement in half a dental arch or 15 sphere units to facilitate placement in a full dental arch.

Thus, according to the second and third embodiments, the matrices are formed as small appropriate anatomically shaped configurations.

Optionally, other embodiments of the present invention may comprise a combination of the embodiments described herein. For example, another embodiment of the present invention may comprise a matrix (10) in the form of a disk, similar to that of the second embodiment, formed integrally with a wedge member, similar to that of the third embodiment, fixed at a circumferential perimeter of the disk. Thus, areas (B1), (B2) and (C) may be dealt with using the same matrix (10) body as a single unit system or a bi or multi layered or a bi or multi located system similar to those illustrated in FIGS. 9b, 9c and 4g. The different sections (i.e. 42 and 46 of FIG. 4b) can also be made of different matrices. The application and activity of this third embodiment of the present invention can be similar to the second embodiment of the present invention.

This system (e.g. FIGS. 4(a) and 4(f)) can be used to protect tooth furcations by inserting the device between the roots of teeth that have experienced periodontal resorption. Further other systems can be bent (e.g. FIG. 3(b)) or cut (e.g. FIG. 6(b) 60′) to fit into a furcation.

In the fourth embodiment of the present invention, the said matrix (10) is in the form of a ribbon or cord which may be impregnated with the active material or materials and which is further adapted for enabling the material or materials to be released in a controlled or sustained manner by intra-oral bio-degradation, chemical activation and/or physical interventions typically heat or light, such as a laser.

In a first configuration of the fourth embodiment, and referring to FIG. 5(a), the matrix is in the form of a ribbon (50) that may be joined—integrally, by means of a knot or in any other manner—in the form of a loop, such as to circumscribe the periphery of a tooth comprising said dental surface and the site that is being targeted for treatment, in particular the enamel dentin and/or cementum at the cervical regions and may also include part of the interproximal regions, as illustrated in FIG. 5(c). The ribbon (50) may be made from any suitable materials, as described herein below. In a second configuration of the fourth embodiment, and referring to FIG. 5(b), the matrix is in the form of a cord (50′) that may be joined—typically by tying together the ends of a length thereof—in the form of a loop such as to circumscribe the periphery of a tooth comprising said dental surface and said interproximal site, as illustrated in FIGS. 5(c) and 5(d). The cord (50′) may be similar to catgut or made from any suitable materials, as described herein below. Both in the first and second configuration of the fourth embodiment the matrix 50 and 50′ are presented as a single uniform matrix but the invention includes other possibilities such as a bi or multi layered or bi or multi-located form similar to those in FIGS. 9b, 9c and 4g.

Typically, in the fourth embodiment, the ribbon (50) or cord (50′) which can be positioned at various other sites such as in the cervical sulcus, may be cut to a required length from a roll thereof, and may vary in size and shape, cross-sectional profile, and so on.

In the fifth embodiment of the present invention, the matrix is in the form of a corset, typically a cervical corset (60) or an occlusal corset (60′) and in the form of straps (62) fixed thereto. These corsets and straps may be made from any suitable materials, as described herein below, and enables the active material or materials contained therein to be released in a sustained or controlled manner to the desired surface, with or without chemical or physical intervention. Although the matrix presented is a single uniform system (60) and (62) the invention includes other possibilities such as a bi or multilayered or bi or multi located form similar to those illustrated in FIGS. 9b, 9c and 4g.

In a first configuration of the fifth embodiment, and referring to FIG. 6(a), a cervical corset (60) is provided for placements around the neck of teeth with or without gingival recession. The cervical corset (60) may be fixed with respect to said dental surface and said site by means of one or more restraining strings or straps (62) which are adapted for securing said corset to a tooth comprising said dental surface. The straps (62) circumscribe at least a portion of said tooth, in particular passing through the interproximal region (B) of the adjacent teeth, as illustrated in FIG. 6(c). The straps are typically made from any suitable material as described herein below, serving as a matrix for delivering the active material or materials to the interproximal site (B), as well as to the cervical site (C).

In the second configuration according to the fifth embodiment of the present invention, and referring to FIG. 6(b), the matrix is provided in the form of an occlusal corset (60′), adapted for fixation at said occlusal site (A) for at least a predetermined time period correlated to the delivery of a predetermined portion of said at least one material to said site, as illustrated in FIG. 6(d). As with the first configuration of the fifth embodiment, the occlusal corset (60′) may be made from any suitable material as described herein below and is typically in the form of a table ribbon. The occlusal corset (60′) may be fixed with respect to said dental surface and said site by means of one or more restraining strings or straps (62). The straps (62) are adapted for securing said corset to a tooth comprising said dental surface, and typically circumscribe at least a portion of said tooth and/or at least a portion of an adjacent tooth, as illustrated in FIG. 6(d). The straps (62) are typically made from any suitable material as described herein below, and also serve as a matrix for delivering the active material or materials to the interproximal site (B).

Thus, in the fifth embodiment, flanking strings or straps containing the active material or materials are tied through the interproximal regions to fluoridate the interproximal regions in addition to retaining the corsets in position. The occlusal corsets (60′) and the cervical corsets (60) may be prepared from long rolled ribbon tapes (with intermittent peripheral holes) that are cut to size for the appropriate usage and can also be shaped to fit the appropriate tooth anatomy, for example. These strips or bands can be positioned on the occlusal surface pit and fissure (FIG. 6(e) to then be altered by a heat or light source in order to seal these pits and fissures. Obviously the strings can also be produced as rolls and the bands outer surface can be made more resistant to degradation than the inner surface.

In the third configuration according to the fifth embodiment of the present invention the matrix is in the form of at least a part of a dental arch which for example covers at least parts of one occlusal, labiallbuccal, palatal/lingual and interproximal site. The system can encompass a part of a dental arch, for example, all surfaces (labial/buccal, palatal/lingual, occlusal) or only one surface, for example, the labial surface of the upper anterior teeth (FIG. 6(i)), and/or an entire dental arch 63 (see FIG. 6(f)) or an upper and lower arch. The system can be soft in order to adapt to the individual arch shape and adhesive to remain in position. The system can also include a separate layer to function as a shield or resorbable or unresorbable tray or the matrix can include properties which simulate at tray. This single matrix system could also be designed to provide a 1-4 minute fluoride tray application within a standard tray or the matrix itself could also function as the tray (FIG. 6g). The system could comprise more than one matrix for example two layers whereby the outer layer (64) could function as unresorbable or a resorbable/biodegradable shield or tray and the inner biodegradable/ resorbable layer (65) provides the active agent or agents FIG. 6(h)). Another example, and referring to FIG. 6(j), is a tooth whitening system whereby the outer layer (64) serves as a shield to protect, for example, the lips, from the active ingredients in the inner layer (65). The shape of the region close to the gingival may be also adapted for protecting the gingival from the active ingredients. The inner layer (65) may be slightly smaller than the outer layer (64) at the gingival margin (69) which may also serve as a means for protecting the gingiva from the active agent or agents.

Optionally, the system according to the second and third embodiments may further comprise specific conventional caries stains and/or light enhanced stains (e.g. fluorescent) which are used without or with blotting agents to draw the stains after the devices are removed and the lesions restoration interface or cracks are flushed with water (see Rawls, H. R., et al., Microbiological Abs Suppl. 261, 1978; Jodaikin, A., et al., J. Oral Path 15, pp 415-418,. 1986. The purpose would be to enhance interproximal caries or leakage diagnosis which remains problematic even with radiographs (see Duncan, R. C. et al. JADA 126:873, 1995) and thus eliminate the need for irradiation.

In a sixth embodiment of the present invention, and referring to FIG. 7(a), the said matrix is in the form of an interproximal “I” device (80) used in conjunction with an orthodontic device such as orthodontic arch wires. The said “I” device (80) may be fixed with respect to said dental surface and said site by positioning under the orthodontic arch wires (90), which were previously installed in the oral, cavity, as illustrated in FIG. 7(b). The vertical potion (82) of the “I” device (80) targets the interproximal areas between the adjacent teeth, while the top and bottom transverse portions (84) are optional, enabling the outer parts of the teeth to be targeted. The said “I” device (80) may be custom made in the appropriate shape, or alternatively made from a ribbon of any suitable material as described herein below and cut out to the required shape and size. The matrix is presented as a single uniform system (80) but the invention includes other possibilities such as a bi- or multi-layered or bi or multi-located form some of which are illustrated in FIGS. 9b, 9c and 4g.

Referring to FIG. 9(a), said matrix (10) may comprise a bi-layer being similar in external structure to the transverse cross-sectional view of the embodiment of FIG. 2(a) taken along x-x. However, the matrix (10) itself consists of a bilayer with two different phases, the internal phase and the external phase. FIG. 9(b) illustrates a bi-layer system similar to the transverse cross-sectional view of the embodiment of FIG. 2(a) taken along x-x. Similarly, FIG. 9(c) illustrates a multi located layer system similar to the cross-sectional view of the embodiment of FIG. 3(a) taken along y-y.

The embodiments described above are not restricted for use with original dental surfaces, and thus the term “dental surface” of a tooth also includes prostheses and restoration margins of a tooth. Thus, as illustrated in FIG. 8, the second configuration of the fourth embodiment and the third configuration of the third embodiment are exemplified with respect to a tooth amalgam (98) or tooth prosthetic crown interface (95).

These applications are not limited to devices, nor are they limited to the treatment regimes described above. For example, they can be placed under gingival flaps of erupting teeth to fluoridate the tooth crown surface or in a tooth furcation to fluoridate, remineralize or mineralize inaccessible root surfaces. In another example, the third embodiment may be used to fluoridate root canals during endodontic and restorative procedures.

These applications are not limited to devices of a biodegradable, resorbable or non-resorbable nature nor any combination thereof which are left in situ, but include devices that are activated or influenced by external means such as chemical or physical intervention, such as laser irradiation with CO₂ lasers, Nd:YAG lasers and Argon lasers.

Again, of course this invention is not limited to the above-described embodiments, but encompasses all the variations thereof. It is also obvious to those schooled in the art that general toxicity, allergic responses and pulp responses need to be investigated prior to applying the proposed techniques clinically.

In the system according to the present invention, the oral activity provided by the active material or materials may be medical treatment such as fluoridization, remineralization or mineralization and desensitization and/or aesthetic treatment such as tooth whitening or providing breath fresheners, and/or any other desired activity.

Thus, the different components of the matrix of the invention can comprise a range of chemicals with the following functions:-

The Primary Active Fluoridating Mineralization and/or Remineralization Agents

The fluoride releasing agent/s and other mineralizing and remineralizing agent/s can be embedded within the polymeric matrix or matrices of the invention, and released from there in a controlled or sustained manner with or without at least one auxiliary chemical or physical step for example laser application to the device in situ. As the matrix or matrices described in this invention may comprise at least one primary active fluoridizing mineralization and/or remineralization agent which provides fluoride and/or other ions, we divide these primary agents into fluoridating agents and other mineralizing and/or remineralizing agents.

Fluoridation Agents

This agent may be any single or any combination of inorganic or organic fluoride-containing pharmaceutically acceptable chemicals known or to be developed. These include, but are not limited to amine fluorides, e.g. olaflur (Ni-octadecyl-trimethylendiamine-N,N,N tris (2-ethanol)-dihydrofluoride) and dectaflur (9-octadecenyl-amine-hydro-fluoride)) hydrofluoride, ammonium fluoride, calcium fluoride, difluorosilane, fluoroaluminosilicate glass and any mixture thereof, hydrogen fluoride, fluoropolymer B (see U.S. Pat. No. 4,837,007), mixed salt neighborite (NaMgF3), magnesium fluoride, magnesium monofluorophosphate, potassium fluoride, lithium fluoride, indium fluoride, zirconium fluoride, copper fluoride, nickel fluoride, palladium fluoride, potassium fluorozirconate, tin fluorozirconate, sodium fluorozirconate, ammonium fluorozirconate, fluorosilicate fluorozirconate, fluoroborates, fluorozirconate, fluorostannites, fluorozirconate, sodium fluoride, stannous fluoride, stannous hexafluorozirconate, sodium hexafluorosilicate, sodium, lithium or potassium monofluorophosphate strontium fluoride and ytterbium trifluoride. Preferably, the active mineralisation agent is sodium fluoride, and/or hydrogen fluoride. This invention is not limited to the above but includes approaches such as the corporation of fluoride in the form of Ca₅ (PO₄ )₃F (see U.S. Pat. No. 4,556,561). Variations in pH and salt types of fluorides (e.g. stannous, ammonium, titanium and amino fluorides) result in different retention of fluoride as calcium fluoride. For example, good results have been obtained using fluoride at lower pH values such as ammonium fluoride (see Jenkins, G. N. The Physiology and Biochemistry of the Mouth p. 495, 1978, Blackwell Scientific Publishing )and preferably thixotropic acidulated phosphate fluoride which can contain about 1-4% sodium fluoride with or without 0.1-0.8% hydrogen fluoride and 0.5-1.5% orthophosphoric acid (see Craig, R. G. et al Dental Materials, Properties and Manipulation p2-28, 2^{nd} Ed 1979 CV Mosby Co.).

The period of fluoride exposure which causes significant rehardening of a demineralized enamel surface is about 4 hours (see Koulourides, T., Art and Science of Dental Caries Research pp. 355--378, 1968; Poole, D. F. G. and Silverstone, L. M., Hard tissue Growth Repair and Remineralisation, pp. 35-52, Ciba Fondation Symposium No.11, Elsevier Scientific Publishing Company, 1973, Pearce E. I. F and Moore, A. J., J. Dent Res 64;416-421, 1985 and references cited in Background to the Invention). Obviously the period of fluoridation required is dependent on the type of material or device herein described, its fluoride type and concentration, frequency and period of delivery, other chemical or physical interventions (such as laser application) and the type of surface or lesion being treated. Furthermore, the effects can also be long term because of the deposition of pH controlled fluoride reservoirs of various CaF₂ forms (see Background to the Invention).

The acute lethal dose of fluoride (F) is 33 mg F/Kg body weight and the chronic toxicity can be 0.1 mg F/Kg. Thus the determination of the fluoride concentration range is governed by the size and number of devices used or the volume of material used, the duration of applying the material or device, the rate of fluoride ion release and the weight of the patient. Thus the concentrations can range from about 7-0.2%. (See: A guide to the use of fluorides JADA 113:504-564, 1986, prepared by the National Fluoride Task force of the NFDH).

Mineralizing and/or Remineralizing Agents

Although fluoride is to date the most effective remineralization agent, this invention and practice thereof is not limited to fluoride alone but may include or be limited to any other mineralizing or remineralization agent known or to be developed or combination thereof. Examples are amorphous minerals, crystalline minerals and organic molecules.

An advantage of an amorphous mineral is that they can be easy to mold into complex shapes (see Levi-Kalisman, Y. et al J. Chem Soc. Dalton Trans 2000: 3977-3982, 2000) such as pits and fissures, demineralized enamel or dentin. These amorphous minerals can be stable or unstable phases. Silica (opal) is a stable type which can be formed by the polymineralization of silicic acid which can be mediated enzymatically. On the other hand amorphous calcium carbonate and amorphous calcium phosphate are unstable as they tend to transform into stable crystalline phases. Amorphous calcium phosphate, amorphous calcium phosphate fluoride, amorphous calcium carbonate phosphate, amorphous calcium carbonate phosphate fluoride, and amorphous calcium fluoride have high solubilities, fast formation rates and fast conversion rates to apatite (see U.S. Pat. No. 5,460,803). This transformation can be controlled, for example by mimicking chiton teeth where amorphous calcium phosphate is converted into dahllite.. Besides these agents there are other agents such as dicalcium phosphate dehydrate which complement fluoride in remineralizing carious lesions. (Wefel, J. S. and Harless, J. D. J. Dent Res 66: 1640-1643, 1987, Takagi, S. et al Caries Res 34: 281-288 (2000)).

Examples of crystalline minerals are aragonite, brushite, (see U.S. Pat. Nos. 3,679,360 and 5,605,677) calcite, dahltite, ferrihydrite, fluoroapatite, hydroxyapatite (which can also be used in dissolved synthetic forms) or in a stannous hydroxy apatite fluoride (see U.S. Pat. No. 4,923,683) , lepidocrocite, magnetite, octocalsium phosphate, vaterite and whitlockite. This invention also includes a system designed to alter a tooth surface thereby enhancing its resistance to caries and other pathology. For example the process of chiton radula formation can be fully or in part adapted to alter tooth surface clinically. For example iron atoms can be introduced which precipitates a hydrated iron-oxide mineral, ferrihydrite which can then be converted to magnetite or an iron oxide mineral, lepidocrocite. Another example is amorphous calcium phosphate can be deposited and then induced to crystallize to dahllite or hydroxyapatite (see Addadi, L. and Weiner, S. Angew, Chem. Int. Ed. Engl. 31:15, 3-169, (1992). Besides hydroxyapatite, an often found mineral at remineralized or mineralize dental sites is whitlockite (Kodaka, T. et al Caries Res 26 : 69-76 (1992). These amorphous or crystalline minerals can be used to restore demineralized tissue such as interproximal caries or to seal regions such as pits and fissures by chemical or physical intervention (such as laser application) to seal areas or alter the chemical surfaces thereof

The organic material can be macromolecules such as acidic proteins, glycoproteins and sulfated polysaccharides (Addadi, L. and Weiner, S. Angew, Chem Int Ed Engl 31:153 169, 1992) or smaller molecules such as polyaspartic and polyglutamic acid with or without a rigid substrate adsorption. (Addadi, L. et al ACS Sym. Series no. 444, 1991).

Enhancing or Other Active Agents

These agents can be the matrix or part thereof or added to the matrix (e.g. silated hydroxyethylcellulose as apatite is formed because silanol chelates calcium (see Turezyn, R. et al J. Biomater Sci. Polym Ed 11:217, 2000.) polyampholyte-sodium fluoride and chlorhexidine (Wefel J. S. et al Am J. Dent. 8,217-220, (1995), (see Caufield, P. W. and Navia, J. M. in the Biological Basis of dental caries, Menaker, L. 406-407, Harper and Row,1980), benzoate-like preserving agents (see Davis, B. A. et al Caries Res 35, 331-337,2001), silanols (see Loty C et al J. Biomed. Mat. Res. 47; 367: 2000), and dicalcium phosphate dihydrate calcium carbonate (see U.S. Pat. No. 4,556,561 and Cury, J. A. et al Caries Res. 183: 2003). Calcium and phosphate are another example (ideally 1,5 m mol/L Ca and 0.9 m mol/L PO4) see Exterkate, R. A. M. et al J. Dent Res. 72:1599-1603, 1993). Examples of suitable calcium compounds are:-calcium chloride, calcium bromide, calcium nitrate, calcium acetate, calcium gluconate, calcium benzoate, calcium glycerophosphate, calcium formate, calcium fumarate, calcium lactate, calcium butyrate, calcium isobutyrate, calcium maleate, calcium maleate, calcium propionate calcium vaerate. Examples of suitable inorganic phosphates are alkali salts and ammonium salts of orthophosphoric acid such as potassium sodium or ammonium orthophosphate, monopotassium phosphate, dipotassium phosphate tripotassium phosphate, monosodium phosphate, disodium phosphate and trisodium phosphate. Other active agents are (e.g. sodium lauryl sulphate (to reduce surface tension), azacycloheptane, diphosphonate, triclosan, polyvinyl methylether with maleic anhydride copolymer (see Zhang et al J. Clin. Dent 14: 23-28 2003) xylitol, vitamin E, aloe vera and rigid beta sheet proteins such as synthetic polyaspartate and polyglutamate proteins and natural agents purified from mineralized tissue such as glycoproteins phosphorylated amino acids and acidic sulfated polysaccharides.(see Addadi et al ACS Symposium series 444; Addadi et al in Chemistry and biology of Mineralized Tissues, Ed. Slavkin, H. and Price, P. Elsevier Sci. Pub. BV 153-162 (1992)), acidic macromolecules associated with hydrophobic macromolecules such as type 1 collagen, alpha and beta chiten (see Addadi, L. and Weiner, s. Angen. Chem. Int. Ed. Engl. 31: 153-169 (1992)) and other molecules such as arginine, silk and elastin. They can also be inorganic agents such as zirconium and ferric pretreatments (see Clarkson B. H. et al. J. Dent. Res. 60:1912-1920 1981) or organic solvents such as urea designed to clean the carious lesion (see Shellis, R. P. et al Eur. J. Oral Sci 110: 392-395, 2002), being part of the system described within the invention or they can be applied prior to the device application.

Acidifying, Buffering or pH Regulating Agents

At least one agent can be included in the matrix or matrices to enhance fluoridation, mineralization or remineralization by altering the pH (3-7) (e.g. acidulated phosphate fluoride (derived from sodium fluoride acidulated with a mixture of sodium phosphate monobasic or dibasic, and phosphoric acid or from sodium fluoride, hydrogen fluoride and orthophosphoric acid), H₃PO₄, citric acid, sodium citrate, or sodium bicarbonate or by inducing buffering with for example calcium carbonate, arginine and polyacrylic acid fully neutralized with alkali metal ammonium or (alkylol) amine compound sodium polyacrylate (see U.S. Pat. No. 6,106,811). Furthermore, buffers may be required to enhance cross-linkage of the matrix or matrices (e.g. phosphate buffers at pH 6.8). Those knowledgeable in the art will know that more than one stage of buffering may be required prior to the production of the final product in order to facilitate required steps such as cross-linking or curing, and optimal pH of the final device which can be low 3-4 for optimal fluoridation remineralization or mineralization or neutral in order not to etch porcelain and tooth colored restorations. Agents which influence pH can also have important roles such as in the case of the remineralization of dentin which have been reported to only occur after the extraction of proteins (see Clarkson, B. H. et al Caries Res 32: 357, 1998). Thus, the matrix or matrices could contain for example lactic acid, acetic acid, phosphoric acid or EDTA in a single matrix or on an external surface layer of a bi or multilayer device. On the other hand the dentin or enamel could be first primed with such agents using a liquid gel or an etching device as described in the present invention, whereby the active agent is an acid, for example 37% phosphoric acid. Such a device could also be used to etch tooth surfaces prior to bonding of dental material. Another type of device could contain both the etching and bonding agent which is activated and/or cured, for example by water and/or light application (I.R., U.V. visual spectrum or lasers). One side of an interproximal device could be inactive and the second side could be an active site which could be used to fill, seal or coat interproximal sites, fissures, pits, lesions, caries, restoration defects or restoration-tooth margin defects. This second side could be a single phase or double phase system.

Another novel approach is the introduction of a buffering agent such as sodium bicarbonate during remineralization which penetrate into the subsurface lesion and then function as a buffering agent during acid challenges (see Tanaka, K. and lijima, Y. J. of Dent. 29: 421-426 (2001)).

The Matrix and Cross-Linking Agents

The role of the matrix or matrices is to carry at least one primary active fluoridation mineralizing or remineralizing agent with or without at least one enhancing agent or other active agent and to provide the required viscosity, strength, plasticity and elasticity for application as well as the required stability or degradation pattern for the delivery of the active and any auxiliary agents, in order to provide the optimal rate and time span of ion or chemical interaction with the tooth surface and to provide a mobile environment for the appropriate ions and/or other chemicals to reach the tooth surface. Those knowledgeable and skilled in the art can alter the degradation by varying the concentrations and the degree of curing or cross-linking and type of cross-linking, or combinations thereof as well as the concentration and types of enzyme inhibitors, antimicrobial agents, preservatives and sterilizing agents which can interfere with intra-oral biodegradation. Some degradation properties may not be required in a matrix or part thereof if specific chemical or physical intervention requires instantaneous delivery.

The types of possible matrices are wide. They can include agents yet unused for dental treatment and agents such as those used as denture adhesives, impression materials, temporary, provisional or permanent restorations, sutures, perio- or surgical packs and periodontal agents (see Dental Therapeutics Digest Odontos Pub Inc.: Kay L. W. Drugs in Dentistry, Bristol 1972; O'Brien, W. J. and Ryge, G. An Outline of Dental Materials, Saunders 1978; Steinberg, D et al., J. Dent Res. 67-208 Abstract No. 767, 1988; U.S. Pat. Nos. 5,324,519, 4,938,763, 5,278,201, 5,077,049, 5,739,176 and 5,733,950). The matrix or matrices material or materials may be sub-classified into natural products and synthetic products.

Polysaccharide polymers (e.g. agar, alginates, carboxymethylcellulose, carrageenan, cellulose, gellan gum, Kelcogel®, Kelcogel®F, Kelco Biopolymers, starches and retted flax extracts), lipids, polyisoprenes (e.g. latex rubber and gutta percha), resins and gums (e.g. tragacanth and storax) and proteins (e.g. alpha or beta chitin, soluble elastin and collagen or denatured collagen in the form of gelatin) are examples of natural products. In some cases agents may need to be dialyzed and de-ionized to remove impurities.

Purified collagen can be untreated or treated with fixing agents to prolong its resistance to digestion (similar to catgut surgical suture production). Denatured collagen can be impregnated with chromium salts to enhance its tensile strength and retard its absorption. A preferred polymeric matrix is a gelatin matrix, although those experienced in the art know the method of dissolution of gelatin is highly technique-sensitive and the method used can cause considerable differences in the texture of the product ranging from jelly-like to thick and ‘ropey’. Further, gelatin, like collagen, can be lysine-cross linked with glutaraldehyde (an organelle preservant which has also been used for human aortic valve implants and dental pulp treatments; Kopel, H. M. et al., J. of Dent, for Child 47: 425-430, 1980) and Periochips®. Another possible cross-linking agent is formaldehyde, which forms intra- and intermolecular methylene bridges between various amino acids. Further examples are allyl methacrylate 2,3, or 3,4, dehydroxybenzaldehyde, glycol dimethacrylate, nordihydroguaiatetic acid, rosemarinic acid, strontium, tannic acid and hexamethylenediisocyanate. Again, the biocompatibility of these agents must be carefully examined even though some of them have been used clinically. The gelatin may be of any source, for example bovine or non-mammalian gelatin. Bovine gelatin is preferably used when a matrix or matrices with higher rigidity is required.

It is prudent to note that a completely natural matrix of gelatin without cross-linking can also be used with an appropriate cover (e.g. support members as described for the first embodiment, which may be composed primarily of 1,4-polyisoprene). Furthermore, natural cross-linkings are also feasible, for example calcium and hydroxylysinorleucine, dihydroxylysinone or leucine (Traub W., and Piez, K., A. Adv. Protein Chem. 25:243-352, 1971) lysine, arginine, proteins polysaccharides such as dextran, lipids such as sodium docusate and dehydrodihydroxylysinonorleucine (Bailey, A. J. et al., Biochem. Biophys. Res. Commun. 35:663-671 1969), and enzymatic cross-linking, for example, by transglutaminase (Orban J. M. et al. J of Biomedical Materials Research 68A:756-762, 2004).

Likely candidates within the boundary of possible synthetic products that may serve for the matrices of this invention are homopolymers or copolymers with a wide molecular weight range formed by condensation, additional anionic, cationic and/or catalytic polymerization systems. Examples are acrylamide based polymers and a cationic monomer (see U.S. Pat. No. 4,837,007) cyanoacrylates, polycarbonates, polyurethane, polyester urethane dimethacrylate, polycaprolactones, ethyl triglycide methacrylate, polysulphides, povidone, polyacrylic methacrylic acid, acrylic and modifications such as poly(hydroxyethyl methacrylate), poly(methylmethacrylate) modified with small amounts of ethyl butyl or other alkyl methacrylates, polyethylene glycol, sodium polyacrylate PEG 400 and PEG 3350 and other carbomers. Some of these are indeed commercial or laboratory products such as carboxymethylcellulose, silated hydroxyethylcellulose or hydroxypropyl methylcellulose (Bourges et al Adv. In Colloid and Interface Sci 215-228: 2002; Bourges X. et al. Biopolymers 63:232-238: 2002) aqueous methacrylic polymer formulations for sustained and controlled release of dental and other products (e.g. Eudragit® Rohm). These polymers may require activators and cross-linking (see below). However, other agents are at times required, for example retarding agents such as hydroquinone and eugenol. Other yet different examples are zinc eugenolate, petrolateum and stearyl alcohol. Other gels may be included such as Carbopol polymers. (BF Goodrich Noveon) or a Na2Si O39H 2 0 solution mixed with phosphoric acid and hydrofluoric acid (see U.S. Pat. No. 3,679,360).

It is to be appreciated that the degree of cross-linking is of major significance to the rate of release of the active and/or auxiliary agents. The determination of the degree of cross-linking of the polymeric matrix or matrices is within the capabilities of the man of skill in the art of pharmacy. Other factors are antimicrobial agents, preservatives, sterilizing agents inhibitors (such as inhibitors of matrix metalloproteinases (see WO 98/16503) and enzyme inhibitors which slow down the biodegradation of the matrix or matrices.

The matrices of the present invention can be strengthened not only by cross-linking, but also by other methods. For Example, U.S. Pat. No. 6,565,960 describes polymer composite compositions in which the polymer fibers, e.g. collagen fibers and gelatin, are strengthened by adding particular catechol-containing compounds, particularly compounds which have two or more catechol groups, to the polymeric material and forming a polymer of the compounds that intercalate within the polymeric material, e.g., forming a polymer composite. According to this U.S. patent, it is possible that the resulting polymer forms a scaffold-like structure throughout the polymeric material without the necessity of cross-linking the individual polymeric materials, e.g., collagen or gelatin polypeptides. This scaffolding provides synthetic polymer fibers having a tensile strength, stiffness, and strain at failure that is comparable to or better than natural polymeric material fibers. As all references cited herein, also U.S. Pat. No. 6,565,960 is fully herein incorporated.

Other novel matrices which can also be used as matrix and sealing agents, for example at pit and fissures, are Sn—Sn catenation, Sn—Cl chains or lattices or Sn protein chains (see Jodaikin, A. and Goldstein, S. J. Dent. 16:140-144, 1988), and even combinations with fluoride, calcium, phosphate and tin (see Harris, N. O. and Christen, A. G. Primary Preventive Dentistry 4^{th} Ed Norwalk Appleton Longe 1995; Wu. H et al abstract from Hua Zi Kou Qiang Yi Xue Za Zhi 18: 219-221, 2000.)

Yet another novelty is a matrix or matrices which is or includes a matrix-bound fluoride ion exchange system which can be ‘recharged’ with fluoride from external sources such as toothpastes, oral rinses, dental materials (see U.S. Pat. No. 5,639,840) and professionally applied fluoride systems (see Zimmerman, B. F. et al J. Dent Res 63:689-692,(1984) Fuji 1X GP® fast by GC Inc.).

Although we have defined the matrix or matrices as a delivery system this invention does not preclude the use of the matrix or matrices itself as a template or framework to control remineralization or mineralization based on control and design principles culled from biological mineralization or fabricated synthetic analogs.

Preservatives and Sterilizing Agents

The addition of preservatives and sterilizing agents may be advantageous particularly for long-dwelling matrices, as they will inhibit the development of various microorganisms such as bacteria, fungi and yeast, and they could play a role in inhibiting the biodegradation of the matrix or matrices, thereby influencing its longevity and the release of the active agent. Examples of preservatives are benzoic acid, biguanide, polyamino propyl, cetyl pyridinium chloride, phenol, methylparaben, metal proteins (see Horman, H. in Sigel, h. Metal Ions in Biological systems Vol 3 New York Marcel and Dekker pg 105, 1974 and Jodaikin, A. and Goldstein, S. J. Dent 16:140-144, 1988), and sodium bicarbonate, sorbic acid, thymol and examples of sterilizing agents are iodine, potassium and alcohol.

Stabilizing Agents

The purpose is to inhibit an unwanted or premature reaction such as reactions of calcium phosphate and fluoride by chemical means or physical means such as the use of a varnishing, coating or encapsulation agent.

Antimicrobial Agents

Included agents for therapeutic functions can be anti bacterial, anti viral, anti fungal and other anti microbial agents. Indeed stannous fluoride has shown antibacterial activity (see Paine, M. L. et al JADA 129, 69-77, 1998). Other examples are alexidine, chlorhexidine digluconate, hexetidine, copper zinc citrate and stannous pyrophosphate, triclosan, cetylpyridinium chloride and halogenated bisphenolic compounds.

Cleaning Agents

In the fourth configuration of the third embodiment the system can function as an interproximal site cleaning system as an alternative or supplement to flossing. The system would thus need to include agents such as a surfactant or sudsing agent which foam throughout a wide pH range. Examples of cleaning agents are sodium alkyl sulfate, sodium lauryl sulfate, sodium coconut monoglyceride sulfonates, sodium lauryl sarcosinate, taurates, sodium lauryl sulfoacetate, sodium lauroyl isothionate, sodium lauryl carboxylate, sodium dodecyl benzenesulfonate, poloxamers, polyoxyethylene sorbitan esters, fatty alcohol ethoxylates, polyethylene oxide, cocamidoppropyl betaine, sodium bicarbonate, monosodiumphosphate, sodium hydroxide, potassium hydroxide, sodium carbonate and imidazole. Another possibility is effervescing agents of systems such as the use of a sodium bicarbonate/citric acid system. The effervescing loosens or dislodges interproximal plaque and debris at a microscopic level thereby overcoming flossing which cannot negotiate rough surfaces, especially at the microscopic level.

Tooth Desensitizing Agents

Examples are fluorides (see above) potassium citrate, potassium chloride, potassium tartrate, potassium bicarbonate, potassium oxalate and potassium nitrate.

Whitening or Bleaching Agents

Although Whitestrips® by Crest have been marketed as a tooth whitening system in the form of a strip which contains hydrogen peroxide this invention includes a system to whiten difficult areas to access such as interproximal regions. The agents that can be used include hydrogen peroxide, carbamide peroxide metal chlorites, perborates, percarbonates, peroxy acids, persulfates, urea peroxide, calcium peroxide, calcium chlorite, barium chlorite, magnesium chlorite, lithium chlorite, sodium chlorite, potassium chlorite, hypochlorite, chlorine dioxide, sodium percarbonate, oxones, and even enzymes such as protease (see U.S. Pat. No. 6,521,215). Stabilizing agents may also be required for example dipicolinic acid or sodium stannate for peroxy bleaching agents.

Anticalculus Agents

Examples are alkalimetal pyrophosphates hypophospite—containing polymers, organic phosphonates, and phosphocitrates. Indeed some anti-calculus agents could enhance anticaries activity and improve fluoride availability (see Zhang, Y. P. et al J. Clin. Dent 14: 23-28, 2003).

Hemostatic Agents

This category includes vasoconstrictors (e.g. adrenalin), absorbable agents (e.g. oxidised cellulose, fibrin, calcium alginate), thromboplastic agents (e.g. thrombin), chemical agents (e.g. tannic acid, ferric chloride, zinc chloride, alum, hydrogen peroxide) or physical plugging (e.g. the device includes bone wax). The role of a hemostat would be to stop bleeding which could hamper fluoridation or chemical treatment in regions where bleeding is caused by gingival or other bleeding.

Liquid Vehicles

Liquid vehicles may be solvents used particularly when preparing the matrix or matrices or to facilitate application. Examples are water, ethyl alcohol or glycerin (glycerol) alone or in any combination.

Plasticisers and Elasticisers

Plasticisers and elasticisers may be used to modify the mechanical properties of the matrix or matrices, where needed and desired. Examples are polyethylene glycol, dibutyl phthalate, glycerol, sorbitol, mineral salts, olive oil, linseed oil, light mineral oil, polymers of ethylene propylene, polyolefins, polyacrylates polymethylates, styrene-butadiene, vinyl ethylene acetate copolymers, butadiene isoprene, gum base, silk and elastin for example, purified from a natural rubbery protein from Ligamentum nuchae).

Another example is carboxypolymethylene which can also be incorporated in the matrix or matrices in order to increase the viscosity of the device and reduce the sorption of saliva thereby also influencing the biodegradation of the device.

According to the first and second embodiments of present invention, the matrix or matrices may be made from any suitable material as described above, such as for example gelatin, in combination with an elasticiser, such as for example soluble elastin, sorbitol or gum base, the gelatin being preferably cross-linked and bound to soluble elastin using any suitable material such as for example glutaraldehyde, nordihydroguaiaretic acid and/or tannic acid. Such matrices have adequate plastic properties and are at the same time of sufficient toughness to maintain the mechanical integrity of the system when affixed within the interproximal space.

According to the third embodiment of the present invention, the matrix or matrices is preferably rigid for the wedge portion, and thus typically lacks the plasticising material of the first embodiment. Nonetheless, the wing members of this embodiment are preferably more elastic and thus may comprise a plasticising and/or elasticising agent.

According to the fourth, fifth and sixth embodiments the matrices, in the form of the corsets, straps and “I” members may be similar as described for the first embodiment mutatis mutandis, with the exception that in these embodiments less elasticising material or none at all may be used in the matrix or matrices.

Adhering Agents

Agents may be added to facilitate adhesion to dental surface. Examples are white wax, bees wax, rosin (colophonium bases) , shellac, mastic and polybutene.

Fillers, Softeners and Binders

The matrix or matrices may also comprise fillers and/or softeners and/or binders such as beeswax, coconut oil, corn syrup, gum Arabic, gum mastic, flour, hydrogenated castor oil, kaolin (aluminum silicate), magnesium oxide, paraffin, silicon dioxide, sodium carboxymethyl-cellulose, xanthan gum, zinc oxide or other various inorganic molecules. It should be noted that certain ions may inhibit remineralization in some cases (for example P₂O₇, HCO₃, SiO₄, CrO₄, Mg and Zn) and some inorganic fillers can be coated with water repellant coupling agents such as vinyl silane. Examples of softeners are lecithin and waxes.

Coloring or Staining Agents

These include agents to enhance the appearance of the applied at least one matrix, and dyes which are released to enhance caries detection, as discussed above. Examples are fuchsin or acid red 52 in propylene glycol. These diagnostic dyes include conventional histological stains, clinical decay detection agents and agents whose detection can be enhanced with light, for example fluorescence agents by UV light or other agents activated by intense light within the visual spectrum, or agents drawn by blotting of the lesion after the device or material is removed and the tooth surface rinsed. A color change system could also be used to indicate for example stages of degradation of the device, pH of the site and/or amounts of fluoride at the site. Another application of coloring is the need for marking of the surface to be treated with a dye in the said device which enhances the effects of lasers such as Nd.Yag (Neodymium-Yttrium Aluminum-Garnet lasers) (see Miller, M, and Truhe, T. JADA 124:32 1993).

Flavoring or Sweetening Agents and Breath Fresheners or Sensates (Warming or Cooling Agents)

A flavoring or sweetening or sensate agent may be added to the matrix or matrices, for example, menthol, sodium saccharin, sorbitol, aspartame, sodium chloride. Also breath fresheners may be added to the matrix or matrices, for example parsley seed, methyl salicylate, sunflower oils and peppermint oil.

It is understood that the invention can include a thickening agent, a sudsing agent, a dessicating agent, an anti plaque agent, an anti-inflammatory agent, humectants, nutrients, an analgesic or anesthetic agent, antioxidants or another therapeutic or cosmetic agent or mixtures thereof.

The matrix or matrices is preferably made from a material, such as for example gelatin cross-linked by glutaraldehyde, nordihydroguaiaretic acid and/or tannic acid that is resorbable and/or biodegradable in the saliva by host enzymes, bacteria or by means of the dissolution properties of the saliva or drinks. Nonetheless, the matrix or matrices may alternatively be made from a non-resorbable material which also releases the active material or materials that is being delivered to the target area. For example, the matrix or matrices may be made from rubber latex, a polymer or any one of a large variety of sugars, lipids, nucleic acids or other proteins found in rubber latex bonded to an amine fluoride which is released in the mouth because of, for example, a host enzyme.

Thus, the present invention provides a method for the prevention and/or treatment of dental caries in a patient in need thereof, comprising applying at a site, such as a cervical root surface, interproximal surface, occlusal surface or carious lesion, of said patient the matrix or matrices according to the invention, wherein the material is a fluoridation agent. Said material is selected from the group consisting of sodium fluoride, stannous fluoride, acidulated phosphate fluoride, calcium fluoride, an amine fluoride, fluoroaluminosilicate glass and any mixture thereof.

Alternatively, the material of the present invention is an amorphous mineral. Said material is selected from the group consisting of amorphous calcium phosphate, amorphous calcium phosphate fluoride, amorphous calcium carbonate phosphate, amorphous calcium carbonate phosphate fluoride, amorphous calcium fluoride and dicalcium phosphate dehydrate.

Alternatively, the material of the present invention is a crystalline mineral. Said material is selected from the group consisting of aragonite, brushite, calcite, dahltite, ferrhydrite, fluoroapatite, hydroxyapatite, lepidocrocite, magnetite, octocalsium phosphate, vaterite and whitlockite

Alternatively, the material of the present invention is made of an organic material. Said material is selected from the group consisting of macromolecules such as acidic proteins, glycoproteins or sulfated polysaccharides, or smaller molecules such as polyaspartic or polyglutamic acid.

Alternatively, the material of the present invention is an enhancing agent or further active agent. Said material is selected from the group consisting of calcium chloride, calcium bromide, calcium nitrate, calcium acetate, calcium gluconate, calcium benzoate, calcium glycerophosphate, calcium formate, calcium fumarate, calcium lactate, calcium butyrate, calcium isobutyrate, calcium malate, calcium maleate, calcium propionate, calcium vaerate, alkali salts, ammonium salts of orthophosphoric acid such as potassium sodium or ammonium orthophosphate, monopotassium phosphate, dipotassium phosphate tripotassium phosphate, monosodium phosphate, disodium phosphate and trisodium phosphate.

Alternatively, the material of the present invention is an acidifying, buffering or pH regulating agent. Said material is selected from the group consisting of acidulated phosphate fluoride, citric acid, sodium citrate, sodium bicarbonate, calcium carbonate, arginine and polyacrylic acid fully neutralized with alkalimetal ammonium or (alkylol) amine compound sodium polyacrylate.

Alternatively, the material of the present invention is an antimicrobial agent. Said material is selected from the group consisting of stannous fluoride, alexidine, chlorhexidine digluconate, hexetidine, copper zinc citrate and stannous pyrophosphate, triclosan, cetylpyridinium chloride and halogenated bisphenolic compounds.

Alternatively, the material of the present invention serves as a cleaning agent. Said material is selected from the group consisting of sodium alkyl sulfate, sodium lauryl sulfate, sodium coconut monoglyceride sulfonates, sodium lauryl sarcosinate, taurates, sodium lauryl sulfoacetate, sodium lauroyl isothionate, sodium laureth carboxylate, sodium dodecyl benzenesulfonate, poloxamers, polyoxyethylene sorbitan esters, fatty alcohol ethoxylates, polyethylene oxide, cocamidoppropyl betaine, sodium bicarbonate, monosodiumphosphate, sodium hydroxide, potassium hydroxide, sodium carbonate and imidazole.

Alternatively, the material of the present invention serves as an effervescing agent. Said material uses a sodium bicarbonate/citric acid system.

Alternatively, the material of the present invention serves as a tooth desensitizing agent. Said material is selected from the group consisting of fluorides, potassium citrate, potassium chloride, potassium tartrate, potassium bicarbonate, potassium oxalate and potassium nitrate.

Alternatively, the material of the present invention serves as a tooth whitening or bleaching agent. Said material is selected from the group consisting of hydrogen peroxide, carbamide peroxide metal chlorites, perborates, percarbonates, peroxyacids, persulfates, urea peroxide, calcium peroxide, calcium chlorite, barium chlorite, magnesium chlorite, lithium chlorite, sodium chlorite, potassium chlorite, hypochlorite, chlorine dioxide, sodium percarbonate, oxones, and protease.

The matrices and devices of this invention and the manufacture thereof are not limited to the above chemical components, but encompass all their variations, and include other chemicals as only examples have been presented above. Further, the biocompatibility of these agents and their interactions need to be carefully examined and tested prior to clinical application.

While in the foregoing description describes in detail only a few specific embodiments of the invention, it will be understood by those skilled in the art that the invention is not limited thereto and that other variations in form and details may be possible without departing from the scope and spirit of the invention herein disclosed or exceeding the scope of the claims.

Aspects of the present invention are further illustrated by way of example. The in vitro fluoride release profile of prototypes was determined using pooled human saliva kept at mouth temperature 37°.

### EXAMPLES

The examples are small round discs, 4,5 mm in diameter, whose weights vary from 0.0010 g to 0.0060 g, and are prepared from solutions and left in plastic plates to dry. The dry sheets are then subjected to a punch to produce the sample discs.

Example 1 consisted of a plate prepared of 1.6 g of marine gelatin heated in 40 ml of distilled water, to which 7 ml of acidulated phosphate fluoride (1.23% F) diluted with 7 ml of distilled water, as well as 1 ml of 0.002% polyamino propyl biguanidine, was added. The solution was then treated with 1 ml of 0.05% glutaraldehyde to cause cross linking. Weight of the dry 5.5 mm diameter disc was 0.0015 g.

Example 2 consisted of a plate prepared from 1.6 g of marine gelatin heated in 40 ml of distilled water, to which 10 ml of saturated sodium fluoride, as well as 1 ml of 0.0002% polyamino propyl biguanidine and 1 ml of 10% glycerin was added. The solution was then treated with 1 ml of 0.05% glutaraldehyde and 1 ml of 0.3% tannic acid to cause cross linking. Weight of the dry 4.5 mm diameter disc was 0.002 g.

Example 3 consisted of a plate prepared from 1.6 g of marine gelatin heated in 40 ml of distilled water, to which 7 ml of acidulated phosphate fluoride (1.23% F) diluted with 7 ml of distilled water, 1 ml of 0.002% polyamino propyl biguanidine as well as 10 ml of 0.01 g of dicalcium pyrophosphate, was added. The solution was then treated with 1 ml of 0.05% glutaraldehyde to cause cross linking. Weight of the dry 4.5 mm diameter disc was 0.003 g.

Control comprised a latent plastic disc which did not contain fluoride.

The samples and control were each placed on a fiberglass grid in 2 ml of fresh pooled human saliva and incubated at 37° for one hour. The subjects had not eaten for 30 minutes prior to being provided with the samples and they had brushed their teeth with a tooth brush without tooth paste. The samples and controls were then removed with the grid, rinsed with 2.5 ml of distilled water and placed in a second container with 2 ml of fresh pooled human saliva and then replaced in the incubator. This procedure was repeated every hour. The residual saliva and 2.5 ml of distilled water used to flush each of the samples were then stored and later measured for fluoride using a fluoride-ion specific electrode in the presence of TISAB buffer according to the manufacturers instructions (Orion model 1720A, Orion Research, Boston Mass., USA). The results of the fluoride release are shown in the graphs in FIGS. 10(a)-10(c) respectively.

The patterns of examples 1, and 3 are similar, showing a peak which falls over the last three hours. Example 2 displays a biphasic release pattern, as discussed above.

The contribution of fluoride from the saliva is negligible and consistent with physiological saliva levels

1. A system for the controlled delivery of at least one material having a predetermined intraoral activity to an interproximal site of at least one dental surface in an oral cavity, comprising at least one matrix containing said at least one material, said at least one matrix being adapted for affixing at said interproximal site for at least a predetermined time period correlated to the delivery of a predetermined portion of said at least one matrix to said site.

2. A system as claimed in claim 1, wherein said interproximal site comprises an area of contact and surrounding surfaces between said dental surface and an adjacent dental surface.

3. A system as claimed in claim 2, wherein said at least one matrix is a polymeric matrix.

4. A system as claimed in claim 3, wherein said at least one matrix comprises a hydrophilic polymer such as to enable said at least one matrix to be fixed by swelling in situ by the hydration thereof in the oral cavity after accommodation at said interproximal site.

5. A system as claimed in claim 3, where said at least one matrix comprise a hydrophilic polymer such as to enable said at least one matrix to be fixed by swelling in situ by the hydration thereof in at least a part of a tooth surface or a carious cavity.

6. A system as claimed in claim 1, in a specific, controlled micro-environment which selectively excludes at least one element or molecule or agent present in the mouth by way of a physical or chemical property of said at least one matrix.

7. A system as claimed in claim 1, in a specific, controlled micro-environment which optimizes the delivery of at least one element, molecule or agent to the said dental site.

8. A system as claimed in any one of claim 1, wherein the system provides at least one agent at a site such as a cervical root surface, interproximal surface occlusally or in a carious lesion to facilitate changes by chemical or physical (such as laser) to prevent, seal, eliminate, retard, treat or heal dental disease, cause desensitization, cleanse, induce whitening or bleaching at a tooth surface or restoration surface.

9. A system as claimed in claim 8, wherein said at least one polymeric matrix has a three dimensional form having an external surface, wherein at least a portion of said external surface is adapted for contact with at least said interproximal site of said dental surface such as to deliver said at least one material to said site.

10. A system as claimed in claim 9, wherein said at least one matrix is in the form of a disc having at least one external substantially flat surface for contact with at least said interproximal site of said dental surface such as to deliver said at least one material to said site.

11. A system as claimed in claim 10, wherein said matrix is attached to a cord, thereby facilitating interproximal placement.

12. A system as claimed in claim 9, wherein said at least one matrix is in the form of a disc having at least one external substantially concave surface for contact with at least said interproximal site of said dental surface such as to deliver said at least one material to said site.

13. A system as claimed in claim 12, wherein said matrix is attached to a cord, thereby facilitating interproximal placement.

14. A system as claimed in claim 9, wherein said at least one matrix is in the form of a pellet having at least one external substantially oval surface for contact with at least said interproximal site and said dental surface such as to deliver said material or materials to said site.

15. A system as claimed in claim 9, wherein said at least one matrix is in the form of a toroidal ring having at least one external substantially annular surface for contact with at least said interproximal site of said dental surface such as to deliver said material or materials to said site.

16. A system as claimed in claim 9, wherein said at least one matrix is in the form of a wedge, having at least one external longitudinal surface for contact with at least said interproximal site of said dental surface such as to deliver said material or materials to said site.

17. A system as claimed in claim 9, wherein said at least one matrix is in the form of a wedge, having at least one winged member at least at one longitudinal end thereof, said wedge having at least one external longitudinal surface for contact with at least said interproximal site of said dental surface such as to deliver said material or materials to said site, and said winged member having suitable contact surfaces for delivering a portion of said material or materials to a portion of said dental surface and an adjacent dental surface mesial and distal to said interproximal site.

18. A system as claimed in claim 9, wherein said at least one matrix is in the form of a sphere, dumb-bell or similarly appropriate shape having at least one surface for contact with at least said interproximal site of said dental surface such as to deliver said material or materials to said site.

19. A system as claimed in claim 18, wherein said at least one matrix is preferably soft for easy interproximal insertion, preferably provides a cleaning effect which would serve as an alternative or supplement to flossing and releases at least one antimicrobial or cleansing agent and/or at least one remineralizing or mineralizing agent.

20. A system as claimed in claim 18, wherein at least one unit is linked to another by a strand or band that can be identical in composition to the units' or it can differ in composition.
